(19) United States
(12) Patent Application Publication
(10) Pub. No.: US 2025/0359812 A1
(43) Pub. Date: Nov. 27, 2025

(54) SYSTEM AND METHODS FOR PROPOSING DETECTION PARAMETERS FOR DETECTING EPILEPTIFORM ACTIVITY

(71) Applicant: NeuroPace, Inc., Mountain View, CA (US)

(72) Inventors: Sharanya Arcot Desai, Mountain View, CA (US); Thomas K. Tcheng, Pleasant Hill, CA (US); Muhammad Furqan Afzal, Forest Hill, NY (US); Brett M. Wingeier, San Francisco, CA (US)

(21) Appl. No.: 19/214,456

(22) Filed: May 21, 2025

Related U.S. Application Data
(60) Provisional application No. 63/650,504, filed on May 22, 2024.

(51) Int. Cl. A61B 5/00; A61B 5/372; A61N 1/36

(52) U.S. Cl. CPC A61B 5/4094; A61B 5/372; A61B 5/7267; A61N 1/36064

(57) ABSTRACT

A method of proposing a detection tool that detects an event in EEG signals sensed by an IMD includes applying a machine learning based model to a plurality of EEG records to identify a set of records with activity indicative of an electrographic seizure. The EEG records comprise a plurality of channel EEG signals sensed by a corresponding plurality of sensing channels of the IMD. The method also includes applying a machine learning based model to the identified set of EEG records to identify channel EEG signals having an earliest seizure onset; and for each of the identified channel EEG signals, processing a plurality of regions of interest to implement a corresponding plurality of candidate detection tools; applying each candidate detection tool to a simulation set of electrographic signals to determine a respective set of metrics; and processing the metrics to identify a selected detection tool from among the candidate detection tools.

### CROSS-REFERENCE TO RELATED APPLICATION

This application claims the benefit of U.S. Provisional Application Ser. No. 63/650,504, entitled “System and Methods for Selecting Detection Parameters for Detecting Electrographic Seizures” and filed on May 22, 2024, which is expressly incorporated by reference herein in its entirety.

### TECHNICAL FIELD

The present disclosure relates generally to methods and systems for proposing detection parameters that enable an implantable medical device to detect an event in physiological data being monitored by the implantable medical device, and more particularly to proposing detection parameters that enable a neurostimulation system to detect epileptiform activity in electrical activity of the brain that is sensed by the system.

### BACKGROUND

Systems and methods that include algorithms for detecting when physiological data sensed from a patient exhibit certain features or correspond to certain physiological states are desirable in diagnosing, monitoring and treating patients. For example, systems and methods that include algorithms for detecting epileptiform activity, which may include pre-ictal epileptiform activity, post-ictal epileptiform activity, inter-ictal epileptiform activity, and/or electrographic seizures, are desirable in diagnosing, monitoring, and treating patients with epilepsy. Specifying the parameters necessary for these algorithms to operate as expected and to generate the desired outcome is generally not an intuitive process for the patient's physician. It would be beneficial to make these systems and methods easier for a physician, clinician, clinical engineer, or other individual involved in the treatment of patients with epilepsy to use with regard to a particular patient or set of patients.

### SUMMARY

This disclosure relates to a method of proposing a detection tool for an implanted medical device (IMD) of a patient, wherein the detection tool detects for an event in EEG signals sensed by the IMD. The method includes applying a machine learning based model to a plurality of EEG records of the patient to identify a set of EEG records with electrographic activity indicative of an electrographic seizure. The EEG records comprise a plurality of channel EEG signals sensed by a corresponding plurality of sensing channels of the IMD. The method also includes applying a machine learning based model to the identified set of EEG records to identify channel EEG signals having an earliest seizure onset; and for each of the identified channel EEG signals, processing each of a plurality of detection windows or regions of interest (ROIs) of the identified channel EEG signal to implement a corresponding plurality of candidate detection tools to detect the event; applying each candidate detection tool of the plurality of candidate detection tools to a simulation set of electrographic signals to determine a respective set of metrics; and processing the respective sets of metrics to identify a selected detection tool from among the candidate detection tools.

This disclosure also relates to a system for proposing a detection tool for an IMD of a patient, wherein the detection tool detects for an event in EEG signals sensed by the IMD. The system includes a memory and a processor coupled to the memory. The processor is configured to apply a machine learning based model to a plurality of EEG records of the patient to identify a set of EEG records with electrographic activity indicative of an electrographic seizure. The EEG records comprise a plurality of channel EEG signals sensed by a corresponding plurality of sensing channels of the IMD. The processor is also configured to apply a machine learning based model to the identified set of EEG records to identify channel EEG signals having an earliest seizure onset; and for each of the identified channel EEG signals, process each of a plurality of detection windows or regions of interest (ROIs) of the identified channel EEG signal to implement a corresponding plurality of candidate detection tools to detect the event; apply each candidate detection tool of the plurality of candidate detection tools to a simulation set of electrographic signals to determine a respective set of metrics; and process the respective sets of metrics to identify a selected detection tool from among the candidate detection tools.

This disclosure also relates to a method of stimulation therapy by an implanted medical device (IMD). The method includes sensing by the IMD, electrical activity of a brain, and applying by the IMD, an electrographic signal corresponding to the electrical activity of the brain to a detection tool selected in accordance with the method describe above, to detect for an event. The method further includes, responsive to a detection of the event by the detection tool, delivering by the IMD, a stimulation therapy to the brain.

This disclosure also relates to an implantable medical device that includes a sensing channel configured to sense electrical activity; a detection tool that is proposed by the system described above and is configured to detect an event in the sensed electrical activity; and a therapy subsystem configured to output a stimulation therapy in response to a detection of the event by the detection tool.

### BRIEF DESCRIPTION OF THE DRAWINGS

Various aspects of apparatuses and methods will now be presented in the detailed description by way of example, and not by way of limitation, with reference to the accompanying drawings, wherein:

FIG. 1A are example time-series waveform visual representations of records of electrical activity, e.g., electroencephalogram (EEG) records, corresponding to electrical activity of the brain recorded by an implanted neurostimulation system, together with their corresponding spectrogram visual representations.

FIG. 1B are example time-series waveform visual representations of EEG records, together with additional information, e.g., time stamps and seizure/non-seizure labels, associated with the records and included in a patient dataset.

FIG. 1C are example time-series waveform visual representations of EEG records corresponding to patterns of electrical brain activity including spikes, oscillatory patterns, and amplitude and/or frequency changes.

FIG. 2 is a block diagram illustration of a system, including an implanted neurostimulation system, a detection proposer, and other external equipment, that enables automatic determination of parameter sets for detection tools of the neurostimulation system based on EEG records recorded by the neurostimulation system.

FIG. 3 is a perspective, schematic illustration of an implanted neurostimulation system implanted in a patient and configured to sense electrical activity of the brain, detect electrographic events through detection tools, and deliver therapy responsive to detections of such events.

FIG. 4 is a block diagram of the implanted neurostimulation system of FIG. 3, illustrating some of the functional subsystems of the system.

FIGS. 5A and 5B is a block diagram illustration of the detection proposer of FIG. 2 that includes an EEG records selection module, a detection window module, a candidate detection tool module, and a detection tool selection module.

FIGS. 6A-6E are further illustrations of the modules of the detection proposer of FIGS. 5A and 5B.

FIGS. 7A, 7B, 7C, and 7D are illustrations of EEG records representing different activity types determined by the detection proposer.

FIGS. 8A-8D is a flow chart of a method of proposing a detection tool for an implanted neurostimulation system using the detection proposer of FIGS. 5A and 5B.

FIG. 9A is a block diagram of a training dataset for training a machine learning based model of the detection proposer.

FIG. 9B is a block diagram of an application of the trained machine learning based model of FIG. 9A to determine candidate detection tools.

FIG. 10 is an illustration of detection tool information and a user interface presented on a display by the detection proposer of FIG. 2.

FIG. 11 is a schematic block diagram of an apparatus corresponding to the detection proposer of FIG. 2.

### DETAILED DESCRIPTION

Disclosed herein are methods and systems for proposing detection parameters that enable a neurostimulation system to detect epileptiform activity, electrographic seizures, and/or other signals characteristic of a neurological disorder in electrical activity of the brain that is sensed by the system. A detection subsystem of the neurostimulation system includes algorithms that are configurable to process electrographic signals and to run one or more algorithms on data corresponding to the signals to decide when characteristics exhibited in the signals should be detected as a condition or an event, e.g., epileptiform activity associated with electrographic onset of an epileptic seizure, that should be recorded or otherwise noted or acted upon. These algorithms are referred to as a “detection tool,” a “detector,” or an “event detector.”

The neurostimulator is configurable to sense electrographic signals obtained from a patient at a predetermined sampling rate and to receive the signals on one or more sensing channels. In some neurostimulation systems, the signals received on each sensing channel are operated on by one or more detection tools to identify characteristics in the data. In some neurostimulation systems, the neurostimulator is provided with any number of detection tools that are configurable by a set of operating parameters, which are also referred to as detection parameters. These detection tools are a half wave detector, a line length detector, and an area detector.

An objective of a half wave detector is to generate an output whenever the power of a portion of a signal falls within a particular frequency range. The particulars of how a half wave detector are configured to operate in the context of a responsive neurostimulation system (or other diagnostic implantable medical device system) are described in more detail below with reference to example(s). Here it is noted generally that, even though a half wave detector can be concerned with the frequency content of a signal, the tool operates in the time domain rather than in the frequency domain.

An objective of the line length detector is to generate an output that corresponds to how much the frequency and/or amplitude of a portion of a signal within a particular time window is varying relative to, for example, a long-term line length trend for that signal. The line length detector is sometimes referred to as a simplification of the fractal dimension of a waveform. The result of the line length detector is meant to correspond to an approximation of the overall power of the signal relative to a trend. For example, the line length detector is meant to “detect” when a portion of a signal in a given time window departs from the trend and exhibits a change in frequency or amplitude swings or both; a change in amplitude or frequency suggests something different is happening in the patient: for example, when the power increases, the line length detector may detect the onset of or a precursor to an electrographic seizure.

An objective of the area detector is to generate an output that corresponds to how much the integral (or area under a curve) of a signal within a particular time window is varying relative to, for example, a long-term area trend for that signal. The area detector is sometimes referred to as a representation of the energy of a waveform. As with the line length detector, the area detector is meant to identify conditions when the signal departs significantly from the long-term trend suggesting something undesirable or abnormal is occurring in the patient.

Even though the half wave detector, the line length detector, and the area detector are each deemed to be algorithms of relatively low complexity, there nonetheless can be a significant number and kind of parameters that need to be specified in order for the running of each algorithm to have an optimal result. A given system can be configured so that all or some of the parameters that control how an algorithm will operate (e.g., what sensed physiological data the algorithm will ‘detect’) are programmable.

The number and kind of parameters for a given detection tool can be relatively easy to understand and specify for an engineer or practicing scientist or for someone who otherwise is interested in how the algorithms operate at a detailed level. However, the typical user (e.g., a busy neurologist or a neurosurgeon with many patients) who is tasked with programming or reprogramming the detection tools may not have the time or inclination to develop a comprehensive understanding of what the various parameters are and how each relates to the condition or state of the patient the user wants the implant to monitor and/or treat. These users are better served by a system that automatically derives the parameters for the various tools based on a dataset of electrographic records.

The complexity of selecting parameters and parameter values for a detection tool are illustrated with reference to the half wave detection tool (or half wave detector). A half wave detection tool is specified by numerous parameters, including: (1) half wave hysteresis; (2) minimum half wave amplitude; (3) maximum half wave amplitude; (4) minimum half wave width; (5) maximum half wave width; (6) half wave count criterion; (7) half wave window size; (8) qualified analysis window count; (9) detection analysis window size, and (10) a persistence parameter. A line length detection tool is specified by numerous parameters, including: (1) a short-term window size parameter, (2) a long-term window size parameter, (3) a detection threshold parameter, (4) a sample count parameter, (5) an inter-sample interval parameter, (6) a threshold logic parameter, (7) a threshold mode parameter, and (8) a persistence parameter.

In the systems and methods disclosed herein, physiological data, e.g., electroencephalogram (EEG) records, acquired from a patient and comprising an electrographic event, e.g., epileptiform activity associated with a seizure onset, are processed automatically to derive a set of parameters and values for the parameters that will be used by one or more detection tools. The intention is that, if the neurostimulator is programmed with the automatically-derived parameter set, then when the relevant detection tool operates on physiological data acquired from the patient in the future, the tool will detect activity of the same electrographic event, e.g., epileptiform activity associated with a seizure onset, if that activity occurs while the tool is being run. The systems and methods therefore automatically specify the detection tool and a set of operating parameters (or detection parameters) for a given detection tool based on EEG records of the patient.

### Datasets/Data Types

As used herein, a “dataset” refers to a collection of information that is used to derive and propose a detection tool. A dataset includes one or more records or files of information from a patient in whom an implantable medical device (IMD) is implanted. This information can include physiological information from the patient and non-physiological information related to the patient's environment, device configuration, device operation, demographics, conditions, and therapies. Physiological information is also referred to herein as data or data types, while non-physiological information is referred to as patient features.

With respect to physiological information, in the case of an implanted neurostimulation system, a dataset includes records or files of physiological information corresponding to electrical activity of the brain that is sensed by the system. Hereinafter, electrical activity of the brain is sometimes referred to as electrographic activity or EEG activity, and a physiological record corresponding to electrical activity of a patient's brain is sometimes referred to as an EEG record. It will be understood that EEG includes electrical activity sensed directly from the neural tissue, which sometimes is referred to as electrocorticographic activity, an electrocorticogram, or “ECOG,” or intra-cranial EEG (“iEEG”).

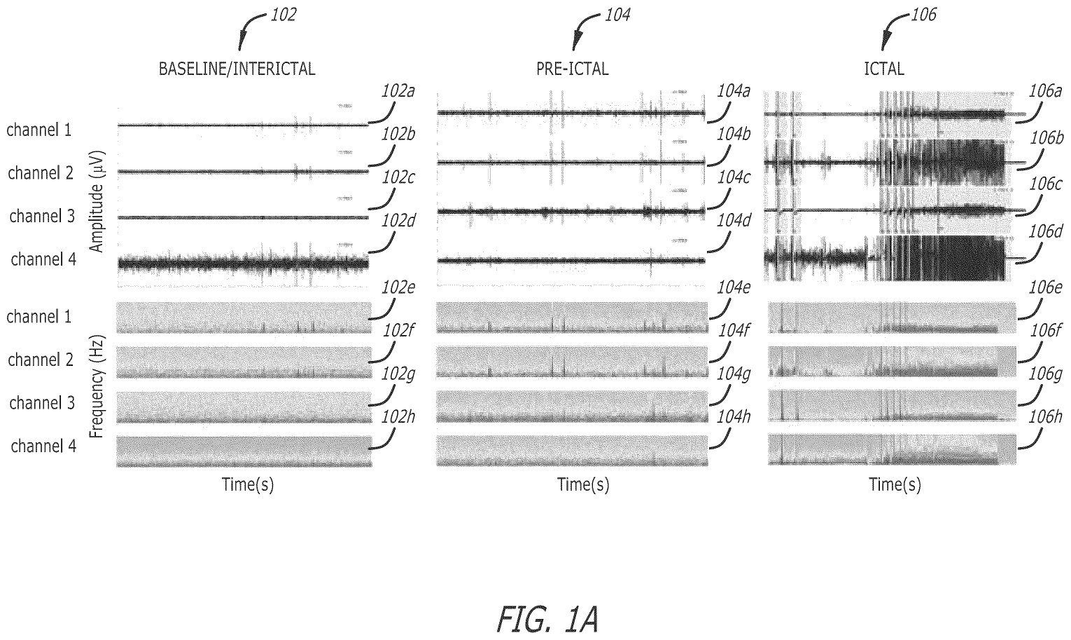

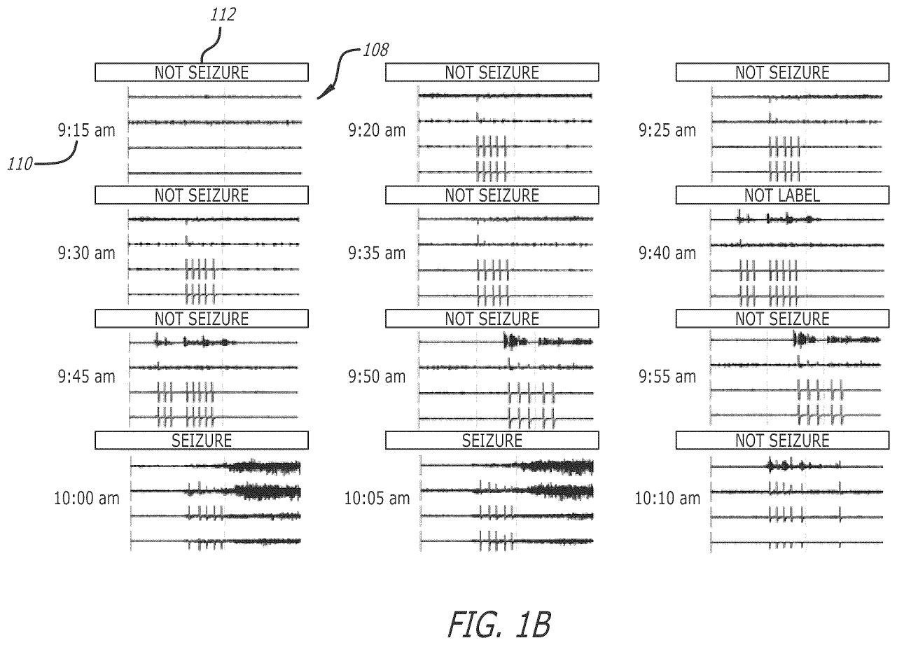

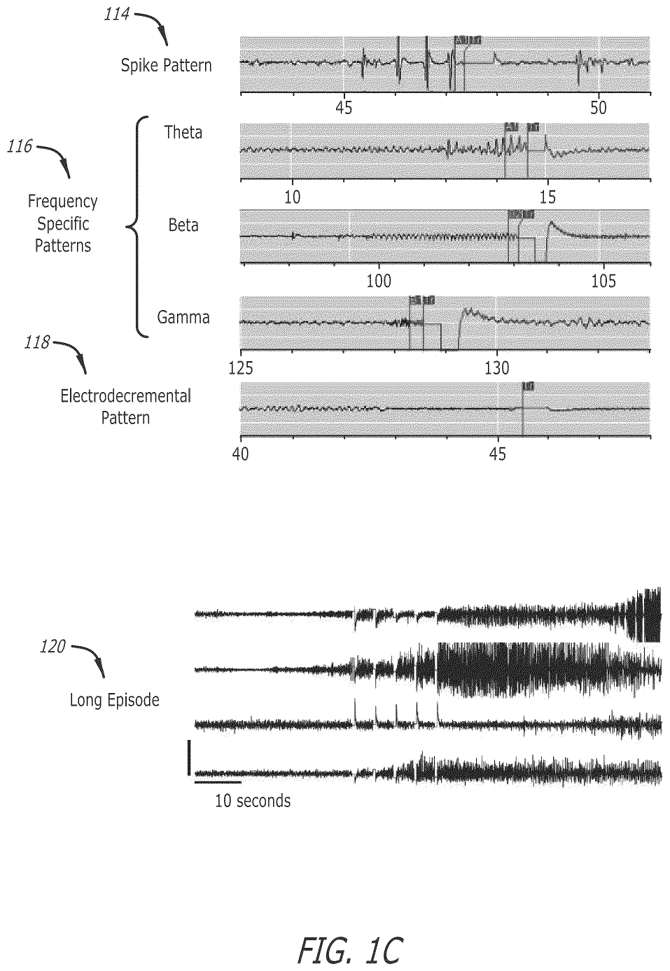

With additional reference to FIG. 1A, EEG records 102, 104, 106 included in a dataset can be visualized or represented in different forms. In the upper portion of FIG. 1A, EEG records 102, 104, 106 are represented by time series waveform images 102a-d, 104a-d, 106a-d for each of four sensing channels of an implanted neurostimulation system. Each EEG record 102, 104, 106 was captured with an implanted neurostimulator system during a respective one of a baseline/interictal brain state (e.g., no seizure), a preictal brain state (e.g., activity captured within the hours before the onset of seizures), and an ictal brain state (e.g., a seizure) in an example patient. In the lower portion, the same EEG records 102, 104, 106 are represented by spectrograms 102e-h, 104e-h, 106e-h for each of the four sensing channels.

With reference to FIG. 1B, additional information or data type can be associated with EEG records. For example, each individual EEG record 108 can have an associated time stamp 110 corresponding to the time the EEG signals within the record were captured by the implanted neurostimulation system. Each individual EEG record 108 can also have an associated label 112 classifying the EEG signals within the record as being indicative of a seizure or not a seizure. Other examples of additional information that can be associated with each EEG record include the event that triggered the creation of the EEG record. As described further below, such triggering events can include a detection of abnormal electrical activity in an EEG signal, a patient-initiated event, e.g., a swipe of a magnet in the area of the implanted neurostimulation system, or a scheduled passage of time.

Additional information or data types can be derived by an implanted neurostimulation system from sensed EEG signals and included in a dataset. For example, in some embodiments, the implanted neurostimulation system is configured to detect patterns in a patient's electrical brain activity and to maintain records of the timing of detections, the count of the number of detections, and a detection rate. The count of such detections can be included in a dataset, either with or without an EEG record of the detected patterns. With reference to FIG. 1C, example patterns of electrical brain activity include spike patterns 114, oscillatory or frequency specific patterns 116, and electrodecremental patterns 118 that exhibit a brief, usually sudden, decrease in the amplitude of brainwave activity. The implanted neurostimulation system can also be configured to detect abnormal electrical brain activity having a duration that exceeds a specified threshold, and to maintain records of the timing and count of the number of such detections together with information, e.g., time stamps, indicative of the time and duration each detection. This abnormal electrical brain activity is referred to as a “long episode.” An example pattern of a long episode 120 is shown in FIG. 1C. The count of detections of long episodes 120 and the respective duration information of each can be included in a dataset, either with or without EEG records of the detected long episodes.

In some embodiments, the implanted neurostimulation system is configured to derive measures from a patient's electrical brain activity and to maintain records of the measures. For example, the implanted neurostimulation system can measure spectral power in certain frequency bands (example 1-4 Hz band, 4-8 Hz band, 8-2 Hz band, 12-25 Hz band, 25-50 Hz band, 50-90 Hz band and so on) computed in small moving and overlapping time windows such as 128, 256 or 512 milliseconds.

While the methods and systems disclosed herein are primarily described with reference to EEG records, it will be appreciated that other physiological information and non-physiological information can be processed. To this end, other types or modalities of physiological information derived from sources other than EEG records can be included in a dataset. For example, physiological records can include measurements of pH level in neural tissue, blood oxygen levels in neural tissue, blood flow rates, neurotransmitters concentrations in neural tissue, temperatures, heart rates, blood pressures, blood glucose levels, hormones sensed in sweat, skin conductivity, accelerometer/motion recordings, posture, and sleep patterns. In some embodiments, this information is sensed and recorded locally by an implanted neurostimulation system. In some embodiments, this information is sensed remote from the implanted medical device, such as from an external wearable device, and transmitted to the implanted neurostimulation system for local storage.

With respect to non-physiological information or patient features, a dataset can include records or files of the patient's demographics (e.g., age, gender, etc.), the patient's drug regimen (e.g., type of drug, dose, and time of day of dose), and the patient's clinical outcomes, such as the rate of clinical seizures (e.g., as reported in a seizure diary), mood, or questionnaire information. A dataset can also include configuration/operation information of the implanted neurostimulation system. Example configuration/operation information includes detection parameters used by the system to detect patterns in a patient's electrical brain activity, and stimulation parameters that define a stimulation therapy delivered by the system.

Typically, some sort of linkage or mapping among the various types of physiological information is provided in a dataset. To this end, in some embodiments each record has one or more associated tags or parameters. For example, physiological records can have a time stamp that allows a set of physiological records at a given point in time to be located for processing. Physiological records can have a tag that indicates the basis, e.g., seizure detection, magnet swipe, scheduled time of day, for preserving the record. These tags allow a set of physiological records to be selected for processing based on a single criterion or a combination of criteria. Other tags include day of capture, area of the brain at which the electrical activity was captured, basis for record creation (e.g., seizure detection, scheduled, patient initiated), characteristic of the record (e.g., power spectral density of EEG signal prior to stimulation).

Examples of data types that can be included in a patient dataset are listed in Table 1:

TABLE 1

| Data Source | Data Type |
|---|---|
| EEG | counts - long episodes (LE), spikes, electrographic |
| (electrical | seizures, abnormal patterns/events |
| activity of | |
| the brain) | |
| | rates - long episodes (LE), spikes, electrographic |
| | seizures, abnormal patterns/events |
| | durations - long episodes (LE), spikes, electrographic |
| | seizures, abnormal patterns/events |
| | distributions - long train distributions (distribution of |
| | episodes that are >10 seconds) |
| | classifications - ictal or interictal |
| | brain activity type (determined by machine learning |
| | based model) |
| | measures - total spectral power, spectral power in certain |
| | frequency bands (example 1-4 Hz band, 4-8 Hz band, 8-2 |
| | Hz band, 12-25 Hz band, 25-50 Hz band, 50-90 Hz |
| | band, phase amplitude coupling, coherence. |
| | spectrogram images |
| | time-series images |
| | coherograms, coherence in seizure activity |
| | coherograms, coherence in scheduled EEG record activity |
| non-EEG | pH level |
| | blood oxygen, flow rate, pressure |
| | temperature |
| | heart rate |
| | neurotransmitter concentrations |
| | glucose level |
| | skin conductivity |
| | hormones sensed |
| | motion/accelerometer |
| | posture |
| | sleep pattern |

Examples of patient features that can be included in a patient dataset are listed in Table 2:

TABLE 2

| Feature Source | Feature |
|---|---|
| patient | age - current, at epilepsy onset, age at implant, sex, |
| demographics | race, etc. |
| | duration of epilepsy (years) |
| | sex |
| | type of job |
| | geographical location |
| patient diagnoses/ | seizure frequency during pre-implant period |
| treatments | |
| | lobe of epilepsy onset - MTL, neocortical, |
| | MTL + neocortical |
| | brain abnormalities - presence of sclerosis, dysplasia |
| | prior cortical resection (yes/no) |
| | prior vagus nerve stimulation (yes/no) |
| | prior EEG monitoring with intracranial electrodes |
| | (yes/no) |
| | drug regimen - list and dosage of medications |
| | clinical outcomes |
| | seizure diary |
| implanted | setting - detection parameters, stimulation parameters |
| neurostimulation | |
| system | |
| | lead implant location |
| | lead type/number - strip, depth |

### Overview of System

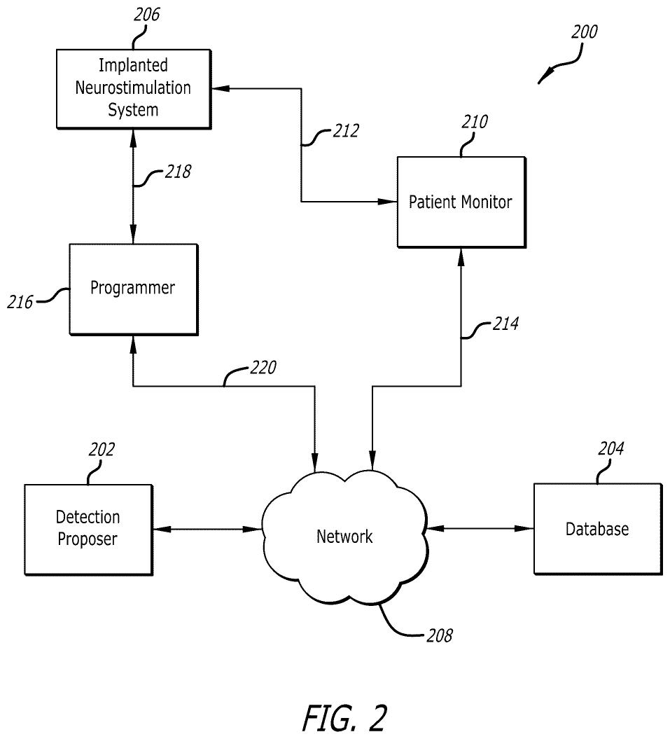

With reference to FIG. 2, a system 200 for processing physiological information, e.g., EEG records, to derive detection tools for a neurostimulation system implanted in a patient, includes a detection proposer 202, a database 204 that stores datasets for processing by the detection proposer, and the implanted neurostimulation system 206 for which one or more detection tools being derived.

The system 200 can also include a patient monitor 210 that interfaces with the implanted neurostimulation system 206 to receive physiological information, e.g., EEG records, from the neurostimulation system 206. The patient monitor 210 also interfaces with the database 204 through a network 208 to provide EEG records and other information to the database, and with the detection proposer 202 through the network 208 to obtain and download to the neurostimulation system 206, the detection tools derived by the detection proposer.

The system 200 also includes a clinician programmer 216 that interfaces with the detection proposer 202 through the network 208. The programmer 216 is configured to obtain information from the detection proposer 202 related to the derivation of the detection tools, and to present the information on the user interface. For example, the information can include the candidate detection sets and proposed detection tools, as determined by the detection proposer 202, and performance metrics, e.g., detection rate, detection lag, etc., for the proposed detection tools when applied to a set of EEG records. The programmer 216 is also configured to receive detection criteria, e.g., desired detection rate, through a user interface and to provide the detection criteria to the detection proposer 202, which criteria is used by the detection proposer 202 to derive detection tools for a neurostimulation system 206. The programmer 216 is also configured to obtain and download to the neurostimulation system 206, the detection tools derived by the detection proposer.

### Implanted Neurostimulation System

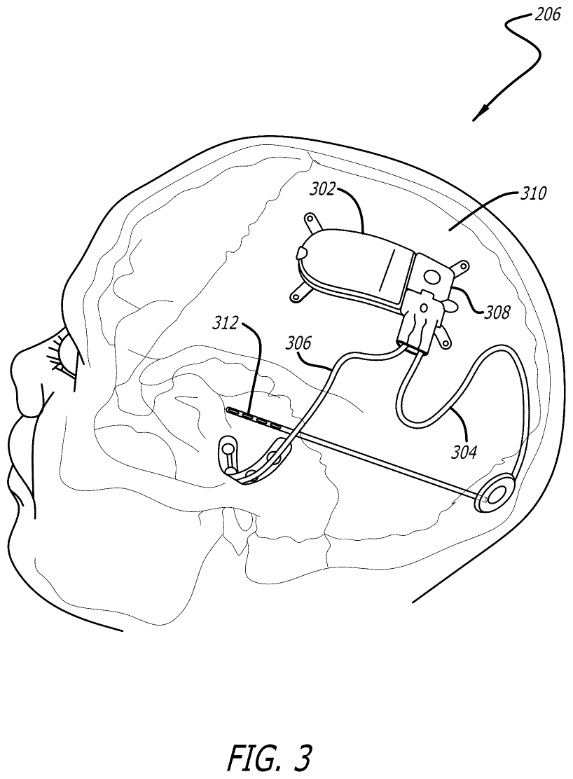

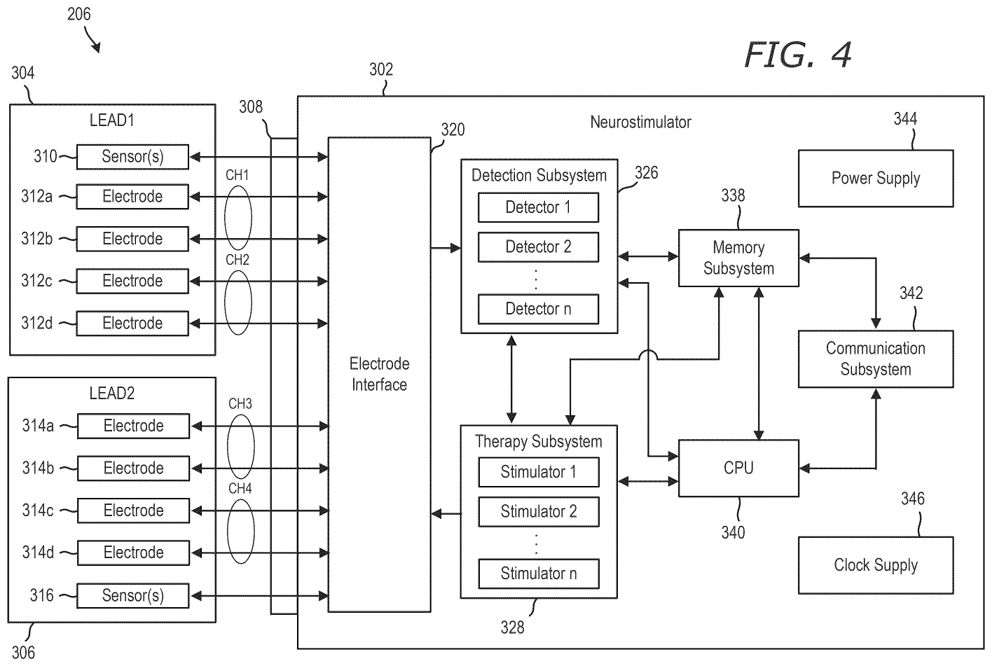

With reference to FIGS. 3 and 4, the implanted neurostimulation system 206 includes a neurostimulator 302 and two electrode-bearing brain leads 304, 306. The neurostimulator 302 includes a lead connector 308 adapted to receive a connector end of the brain leads 304, 306, to electrically couple each lead and its associated electrodes 312a-d, 314a-d with the neurostimulator. The brain leads include a depth lead 304 and a cortical strip lead 306. The depth lead 304 is implanted so that a distal end of it is situated within the patient's neural tissue, whereas the cortical strip lead 306 is implanted under the dura mater so that a distal end of it rests on a surface of the brain. In some embodiments the brain leads may include leads whose distal form factor is furcated, gridded, helical, or planar. In some embodiments the brain leads may include leads whose distal end is situated in or substantially in a cerebral ventricle, a blood vessel, the epidural space, the subgaleal space, and/or the volume of the skull between inner table and outer table. In some embodiments, the neurostimulator 302 is positioned near the patient's neural tissue, for example intracalvarially or in a full- or partial-thickness craniotomy, and includes one or more conductive elements, such as an electrically active metallic outer casing, in lieu of one or more brain leads.

The neurostimulator 302 can configure one or more channels, each comprising a pair of the electrodes 312a-d, 314a-d, as either a sensing channel (for purposes of sensing electrical activity of the brain) or a stimulation channel (for purposes of delivering therapy to the patient in the form of electrical stimulation) or both. To these ends, the electrodes 312a-d, 314a-d are connected to an electrode interface 320 that is configured to form these channels. The electrode interface 320 also provides other features, capabilities, or aspects, including but not limited to amplification, isolation, and charge-balancing functions, that are required for a proper interface with neurological tissue.

With respect to sensing channels, the electrode interface 320 is configured to form a sensing channel by coupling a pair of electrodes to a detection subsystem 326 that includes one or more detectors configured to process electrical activity of the brain sensed through the pair of electrode 312a-d, 314a-d. Each detector is applied to a sensing channel, which is coupled to a pair of electrodes. In some cases, two or more detectors are applied in parallel to a sensing channel. Each detector is defined by a set of detection parameters to detect a particular event, e.g., abnormal electrographic activity, an electrographic seizure, or the onset of an electrographic seizure, of a particular type, e.g., low voltage fast, hypersynchronous, etc., in a sensed EEG.

For example, a half wave detector can require specifying several parameters, including: (1) half wave hysteresis; (2) minimum half wave amplitude; (3) maximum half wave amplitude; (4) minimum half wave width; (5) maximum half wave width; (6) half wave count criterion; (7) half wave window size; (8) qualified analysis window count; (9) detection analysis window size, and (10) a persistence parameter. A line length detector can require a minimum of eight parameters to be specified, including: (1) a short-term window size parameter, (2) a long-term window size parameter, (3) a detection threshold parameter, (4) a sample count parameter, (5) an inter-sample interval parameter, (6) a threshold logic parameter, (7) a threshold mode parameter, and (8) a persistence parameter.

The neurostimulation system 206 is configured to generate records of electrical activity based on an occurrence of an event detected by a detection tool, or an occurrence of a trigger. To this end, the neurostimulation system 206 can be configured to create an EEG record of a sensed EEG when an event, e.g., an electrographic seizure or the onset of an electrographic seizure, is detected by a detection tool, and to create an EEG record of the corresponding EEG signal spanning the time period 60 seconds before the event was detected and 30 seconds thereafter. The neurostimulation system 206 can also be programmed to create an EEG record of a sensed EEG at certain times of day (e.g., at noon and at midnight). These are sometimes referred to as “scheduled EEGs.” In addition, the neurostimulation system 206 can be configured to store an EEG record upon some other trigger, such as when the patient swipes a magnet over the location on the patient's body at which the neurostimulator is implanted (the patient might be instructed to do this whenever he or she thinks a seizure is coming on).

In some embodiments, the neurostimulation system 206 is programmed to designate EEG records based on the event that triggered its recording and to include that designation in the EEG record. For example, EEG records resulting from the detection of abnormal electrical activity, e.g., an electrographic seizure or the onset of an electrographic seizure, are marked as such, while EEG records that do not reflect abnormal activity are designated as baseline EEG records. Thus, for a given patient, a dataset can contain EEG records corresponding to what is happening in the patient's brain during and around when an event occurs, scheduled EEG records acquired at a particular time, and EEG records stored by the neurostimulator when a patient triggers storage with a magnet. Some of these EEG records, especially the ones recorded at the time of an event or when triggered by a magnet swipe, may reflect the patient's electrographic seizures. The dataset can include information or a data type about whatever triggered the neurostimulator to store a given EEG, such as the type of event (e.g., Pattern “A” or Pattern “B,” a magnet swipe) or the time of day (e.g., scheduled EEG).

In some embodiments, the neurostimulation system 206 is configured to capture different data types based on EEG signals. Data types can be captured at different time scales. Some examples of data types captured by a neurostimulation system 206 include: (1) continuous recordings (EEG records) of raw brain data at a certain sampling rate such as 1000, 500 or 250 Hz, (2) continuous measures of derived brain data such as spectral power in certain frequency bands (example 1-4 Hz band, 4-8 Hz band, 8-2 Hz band, 12-25 Hz band, 25-50 Hz band, 50-90 Hz band and so on) computed in small moving and overlapping time windows such as 128, 256 or 512 milliseconds; (3) counts of abnormal events in bins of varying durations such as minutes, days or hours; (4) sampled raw time series or derived brain data that are saved at random time points, specific time points (preprogrammed by a physician for example) or are sampled in response to a trigger such as detection of abnormal events in brain or when a patient swipes a magnet over the neurostimulator; and (5) patient reports of outcomes. These are almost always not continuous and only intermittently available.

With respect to stimulation channels, the electrode interface 320 is configured to form one or more stimulation channels by coupling a pair of electrodes to a therapy subsystem 328, which is configured to deliver electrical stimulation therapy to the patient through the electrode 312a-d, 314a-d. The therapy subsystem 328 includes one or more stimulators, each defined by a set of stimulation parameters to deliver electrical stimulation therapy in response to “events” detected by the detection subsystem 326. The stimulation parameters can include: (1) a stimulation path that defines the electrodes through which stimulation is delivered, (2) a pulse width in microseconds, (3) a pulse frequency in Hz, (4) a pulse current in milliamps, (5) a burst duration in seconds, and (6) a pulse charge density, which is calculated from current, pulse width, and electrode surface area.

In some embodiments, one or both of the brain leads 304, 306 have one or more physiological sensors 310, 316 that enable the capture and recording of other types of physiological information, e.g., pH levels, blood oxygen levels, neurotransmitters concentrations, heart rate, blood pressure, blood glucose levels, hormone levels, sleep states, posture, etc. To this end, one or both of the brain leads 304, 306 is configured as disclosed in U.S. Pat. No. 10,123,717, entitled Multimodal Brain Sensing Lead, which is herein incorporated by reference, and the one or more physiological sensors 310, 316 correspond to different transducers, e.g., macroelectrodes, microelectrodes, light emitters, and photodetectors that enable different sensing modalities.

In some embodiments, the neurostimulation system 206 includes one or more electrodes configured to sense electrical cardiac activity indicative of heart rate, a pressure sensor configured to provide signals indicative of blood pressure, an accelerometer configured to provide motion signals indicative of motion and the position of the patient. From these accelerometer signals, the implanted neurostimulation system 206 can derive other physiological information corresponding to clinical seizures, patient posture, and sleep state.

In some embodiments, other types of physiological information is obtained and stored by the neurostimulation system 206 from sources independent of the neurostimulation system. For example, an external wearable device, e.g., patch, can include a sensor configured to sense and track cortisol levels, i.e., stress hormones, in sweat, while an external wearable device, e.g., watch, can include or a sensor configured to sense blood pressure. The physiological information from these external devices is transmitted to the implanted neurostimulation system 206 for inclusion in the patient's dataset.

With reference to FIG. 4, the neurostimulator 302 includes a memory subsystem 338 and a central processing unit (CPU) 340, which can take the form of a microcontroller. The memory subsystem 338 is coupled to the detection subsystem 326, and receives and stores records of data representative of sensed electrographic signals for subsequent transmission to the database 204. The memory subsystem 338 is also coupled to the therapy subsystem 328 and the CPU 340.

The neurostimulator 302 also includes a communication subsystem 342. The communication subsystem 342 enables communication between the neurostimulator 302 and an external device, such as a programmer 216 or patient monitor 210, through a wireless communication link. The neurostimulator 302 also includes a power supply 344 and a clock supply 346. The power supply 344 supplies the voltages and currents necessary for each of the other subsystems. The clock supply 346 supplies substantially all the other subsystems with any clock and timing signals necessary for their operation.

### Detection Proposer

The detection proposer disclosed herein is configured to: 1) classify EEG records sensed by an implanted medical device into seizure EEG records and non-seizure EEG records, 2) to group and process seizure EEG records based on seizure onset times and electrographic activity types to determine candidate sets of detection parameters, also referred to as “candidate detection sets,” 3) to build and test detection tools based on the candidate detection sets, and 4) to select one or more detection tools for programming into the implanted medical device based on the outcome of the tests.

Example detection tools include the above-described half wave detector, line length detector and an area detector. As previously mentioned, a detection set, also referred to as a parameter set, for a half wave tool can include: (1) half wave hysteresis; (2) minimum half wave amplitude; (3) maximum half wave amplitude; (4) minimum half wave width; (5) maximum half wave width; (6) half wave count criterion; (7) half wave window size; (8) qualified analysis window count; (9) detection analysis window size, and (10) a persistence parameter. A detection set for a line length tool can include: (1) a short-term window size parameter, (2) a long-term window size parameter, (3) a detection threshold parameter, (4) a sample count parameter, (5) an inter-sample interval parameter, (6) a threshold logic parameter, (7) a threshold mode parameter, and (8) a persistence parameter.

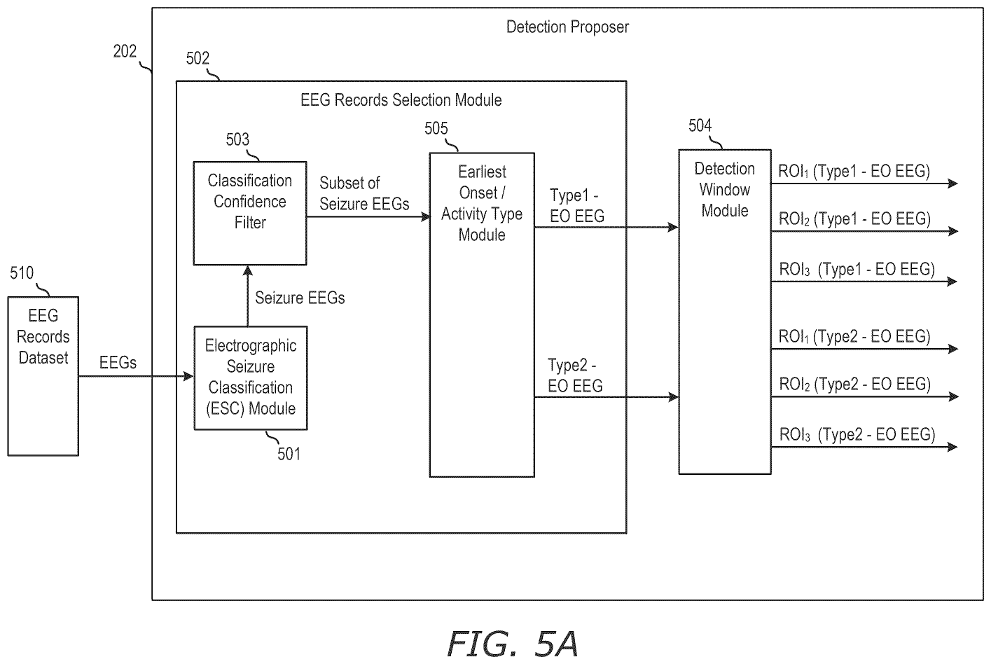

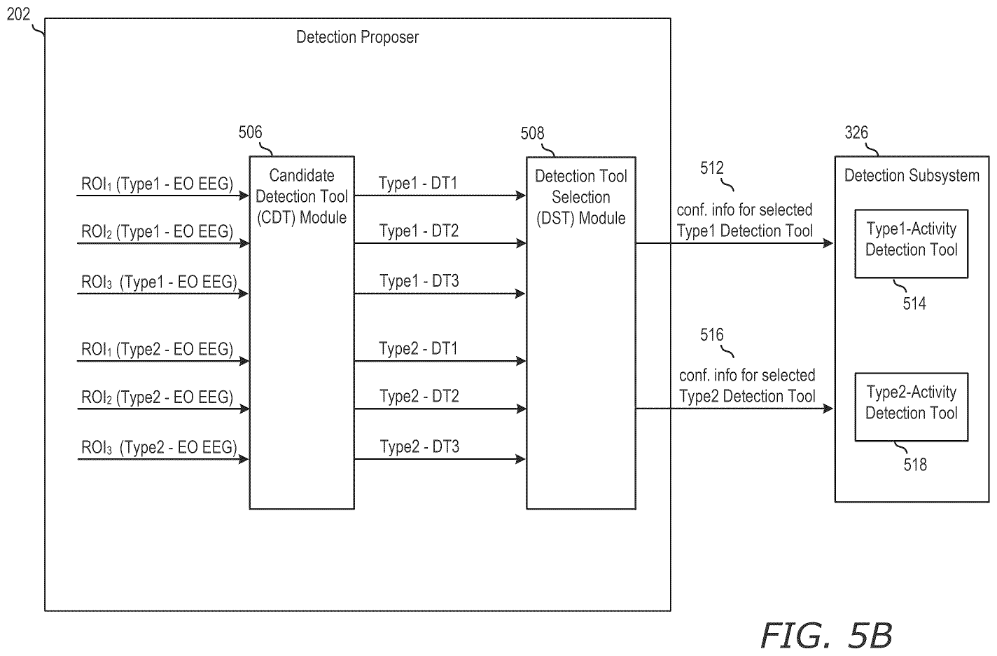

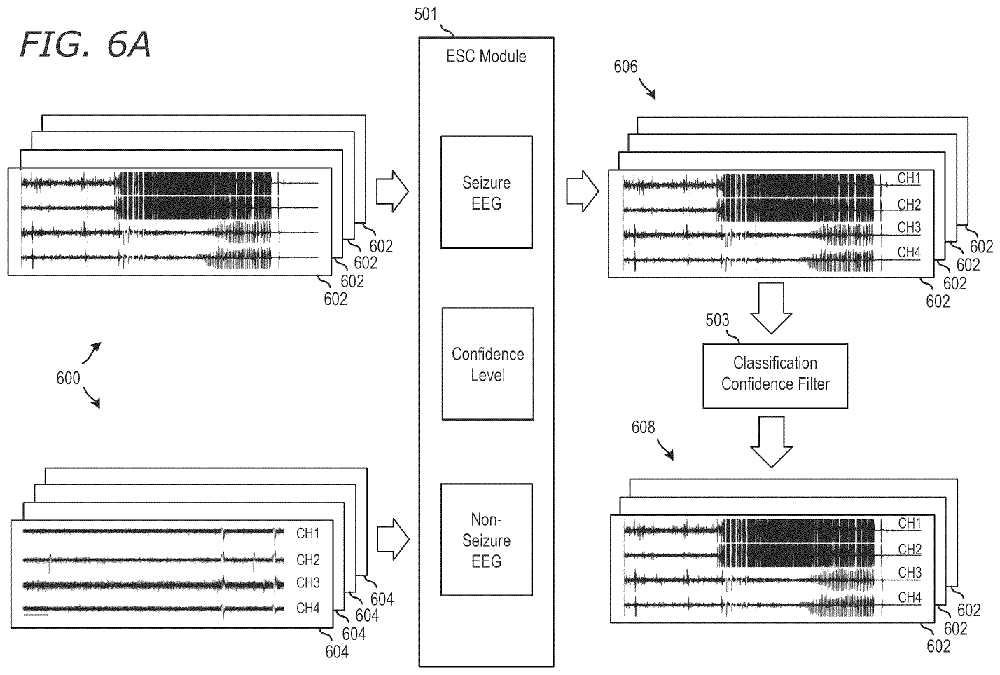

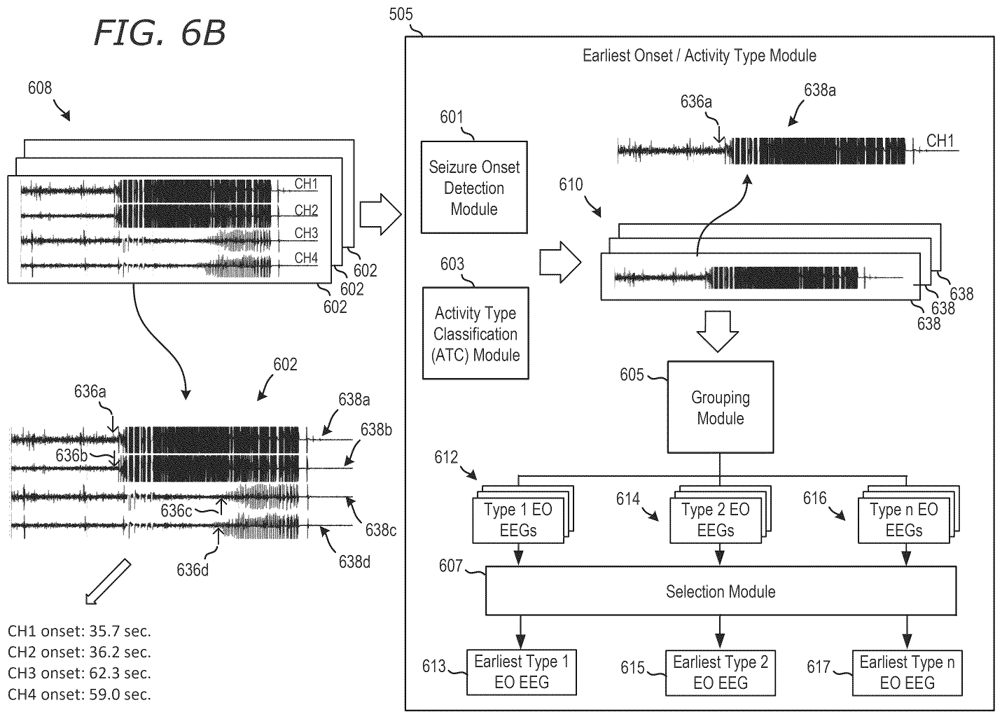

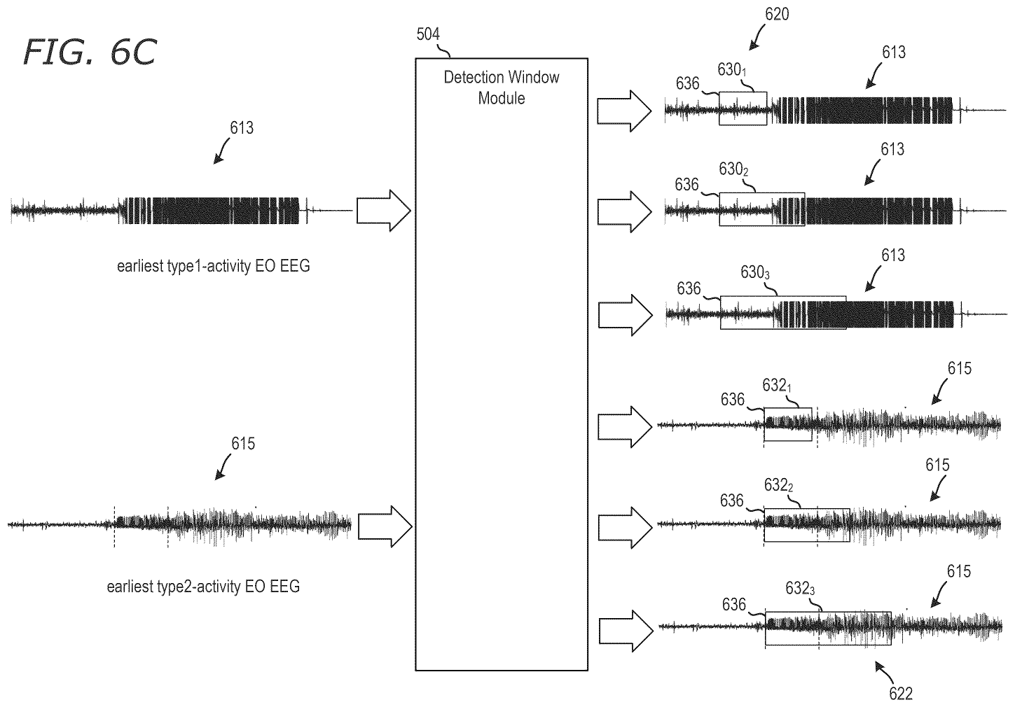

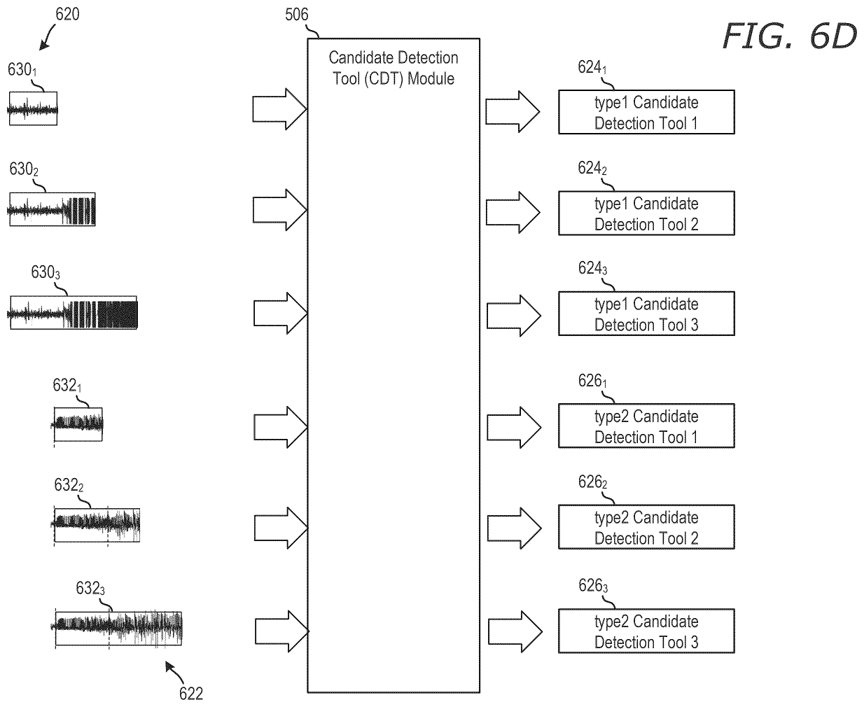

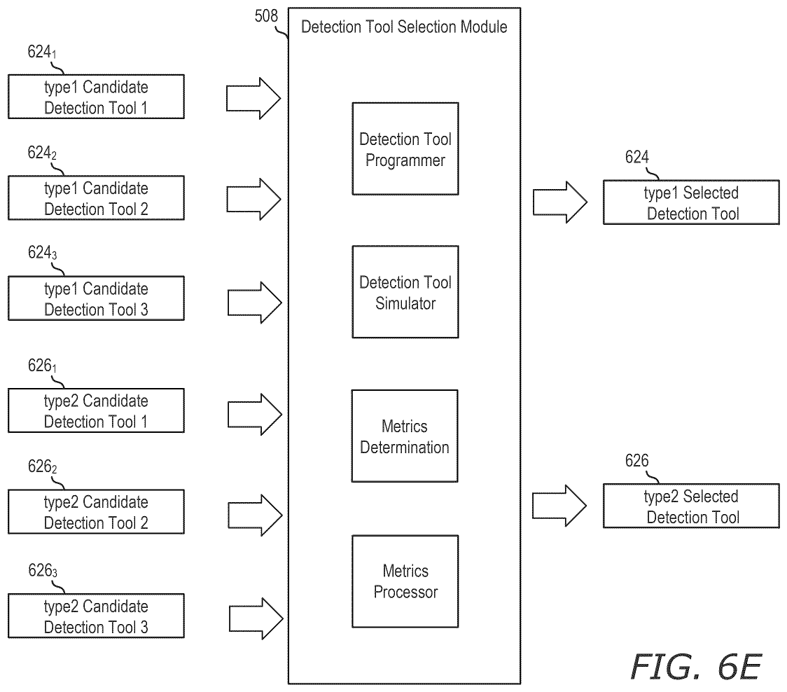

With reference to FIGS. 5A and 5B, in an example configuration the detection proposer 202 includes an EEG records selection module 502, a detection window module 504, a candidate detection tool module 506, and a detection tool selection module 508. The EEG records selection module 502 includes an electrographic seizure classification (ESC) module 501 (also referred to as an ESC model), a classification confidence filter 503, and an earliest-onset/activity-type (EO/AT) module 505 (also referred to as an EO/AT model). Additional modules and functionalities of the EEG records selection module 502, the detection window module 504, the candidate detection tool module 506, and the detection tool selection module 508 are disclosed below with reference to FIGS. 6A-6E.

As shown in FIG. 5A, the detection proposer 202 is configured to interface with an EEG records dataset 510, which can be part of the database 204 of FIG. 2, for purposes of receiving EEG records for processing. The detection proposer 202 also interfaces with a display (not shown) to enable the display of EEG records, the display of configuration information (e.g., detection tool type, detections parameter set) for candidate detection tools and selected detection tools, as determined by the detection proposer, and the display of metrics of detection tool performance, such as detection rate, detection lag, and false positive detection rate. The detection proposer 202 can also interface with a user interface (not shown) to receive inputs from a user that indicate a desired value for a metric. For example, a user can input a desired detection rate for a detection tool. Based on this input, the detection proposer 202 can select, from among a number of candidate detection tools, the tool having a detection rate that matches (or is within a tolerance of matching) the desired detection rate.

With reference to FIGS. 5A and 6A, in an operation of the detection proposer 202, the ESC model 501 of the EEG records selection module 502 is applied to a set 600 of multichannel EEG records (in this case the EEG records includes four channels: CH1, CH2, CH3, and CH4). The ESC model 501 is configured to classify each multichannel EEG record in the set 600 of records as either a seizure record 602 or a non-seizure record 604. The ESC model 501 is also configured to assign a confidence level that represents the likelihood that a multichannel EEG record 602 classified as a seizure record is indeed a seizure record. In one example, the ESC model 501 assigns a confidence level in the range of 0.1 to 0.9, where 0.1 indicates a low confidence level and 0.9 indicates a high confidence level.

In some embodiments, the ESC model 501 is a machine learning based model trained in accordance with one of the models or a combination of models described in Barry W, Arcot Desai S, Tcheng TK and Morrell MJ (2021), A High Accuracy Electrographic Seizure Classifier Trained Using Semi-Supervised Labeling Applied to a Large Spectrogram Dataset. Front. Neurosci. June 2021, Vol. 15, Art. 667373 (hereinafter referred to as Barry et al.), and/or Arcot Desai S, Afzal MF, Barry W, Kuo J, Benard S, Traner C, Tcheng T, Seale C and Morrell M (2023), Expert and deep learning model identification of iEEG seizures and seizure onset times. Front. Neurosci. July 2023, 17:1156838 (hereinafter referred to as Arcot Desai et al.), each of which is incorporated by reference. Also see Leveraging Artificial Intelligence Models for Classifying Electrographic Seizures and Identifying Seizure Onset Times in Patients Treated with the RNS System, AES abstract no. 2.186, published Dec. 8, 2024.

For example, a large dataset of EEG records from over a hundred patients can be manually labeled as seizures and non-seizures and used to train an attention-based ESC model 501 based on a convolutional neural network (CNN). One such trained model described in Barry et al. had a test classification accuracy of >95% on individual EEG channels which means the trained model could classify EEG channels from new patients as seizures and non-seizures with over 95% classification accuracy. In some embodiments, the attention-based ESC model 501 is based on a vision transformer architecture (ViT) or other neural network architecture.

With continued reference to FIG. 6A, a set 606 of multichannel seizure records 602 is applied to the classification confidence filter 503. The classification confidence filter 503 is configured to filter the set 606 of multichannel seizure records 602 based on a threshold confidence level to provide a subset 608 of multichannel seizure records 602 for further processing by the earliest-onset/activity-type module 505. In some embodiments, the classification confidence filter 503 is programmed to include multichannel seizure records 602 with a confidence level at or above the threshold confidence level in the subset 608 of seizure records. The threshold confidence level can be, for example, 0.8.

With reference to FIGS. 5A and 6B, a seizure onset detection (SOD) module 601 (also referred to as an SOD model) of the earliest-onset/activity-type module 505 is applied to the subset 608 of multichannel EEG records 602. The SOD module 601 is configured to predict a seizure onset 636a-d for each channel EEG record 638a-d in a multichannel seizure record 602 and to provide a corresponding seizure onset time. For example, in FIG. 6B, the CH1 EEG record 638a is determined to have a seizure onset 636a at a time of 35.7 seconds, the CH2 EEG record 638b is determined to have a seizure onset 636b at a time of 36.2 seconds, the CH3 EEG record 638c is determined to have a seizure onset 636c at a time of 62.3 seconds, and the CH4 EEG record 638d is determined to have a seizure onset 636d at a time of 59.0 seconds.

In some embodiments, the SOD module 601 comprises a convolutional neural network (CNN) based architecture. In this architecture, a large dataset of EEG records is manually labeled for seizure onset times on individual EEG channels and a CNN is trained on the labeled dataset. See, for example, Arcot Desai et al. The trained CNN is applied to a multichannel seizure record 602 to predict seizure onsets and provide times of seizure onsets for each channel in the record. In other embodiments, the SOD module 601 is a deep learning model created using different methods, such as attention models (vision transformers) or signal processing algorithms. See, for example, “Recurrent Neural Networks for Forecasting Epileptiform Electrographic Activity 24 Hours in Advance,” AES abstract no. 2.050, published Nov. 6, 2018. Also see Leveraging Artificial Intelligence Models for Classifying Electrographic Seizures and Identifying Seizure Onset Times in Patients Treated with the RNS System, AES abstract no. 2.186, published Dec. 8, 2024.

In some embodiments, the earliest-onset/activity-type module 505 includes an activity type classification (ATC) module 603 (also referred to as an ATC model) configured to classify seizure EEG records as a type of seizure onset. In one configuration, the ATC module 603 invokes a clustering method in which features are extracted from the EEG records and passed through a clustering algorithm such as Bayesian Gaussian Mixture Models as disclosed in Barry et al. to determine which type/pattern (or cluster) of seizure onset the EEG records falls within. The ATC module 603 is trained to recognize various type/patterns of electrical activity in EEG records corresponding to seizure onsets. Some examples are shown in Table 3 below along with a description. EEG examples with these activity patterns are further described in Nune et al., “Treatment of drug-resistant epilepsy in patients with periventricular nodular heterotopia using RNS System: Efficacy and description of chronic electrophysiological recordings,” Clinical Neurophysiology 2019, which is incorporated by reference.

TABLE 3

| Seizure Onset Type | Description |
|---|---|
| Low Voltage Fast | Slowly evolving, high frequency (>13 Hz) and low |
| (LVF) | amplitude activity progressing to lower frequencies |
| | and higher amplitude |
| Hypersynchronous | Periodic spiking at less than 3 Hz for a duration |
| (Hypersync) | of >10 seconds prior to seizure onset |
| Attenuation (Atten) | Voltage suppression without a significant increase |
| | in power at any frequency |
| Multiple (M) | Multiple concurrent onset types |
| Rhythmic Delta (D) | Rhythmic activity at 1 to <4 Hz or periodic spiking |
| | at >3 and <4 Hz |
| Rhythmic Theta (T) | Rhythmic 4 to <8 Hz activity |
| Rhythmic Alpha (A) | Rhythmic 8 Hz to <13 Hz activity |
| Semi-Rhythmic | High but variable amplitude, semi-rhythmic spiking |
| Beta (SRB) | in the beta frequency range |

As an alternative to a clustering algorithm, the ATC module 603 can invoke a rule based method in which features extracted from the EEG records are used to classify a seizure based on pre-set rules. Such rule based methods are disclosed in U.S. Pat. No. 10,543,368, “Seizure Onset Classification and Stimulation Parameter Selection,” which is incorporated by reference. Examples of features extracted from EEG records include spectral power in delta, theta, alpha, beta, low gamma and high gamma bands. A list of other features is disclosed in U.S. Pat. No. 10,543,368.

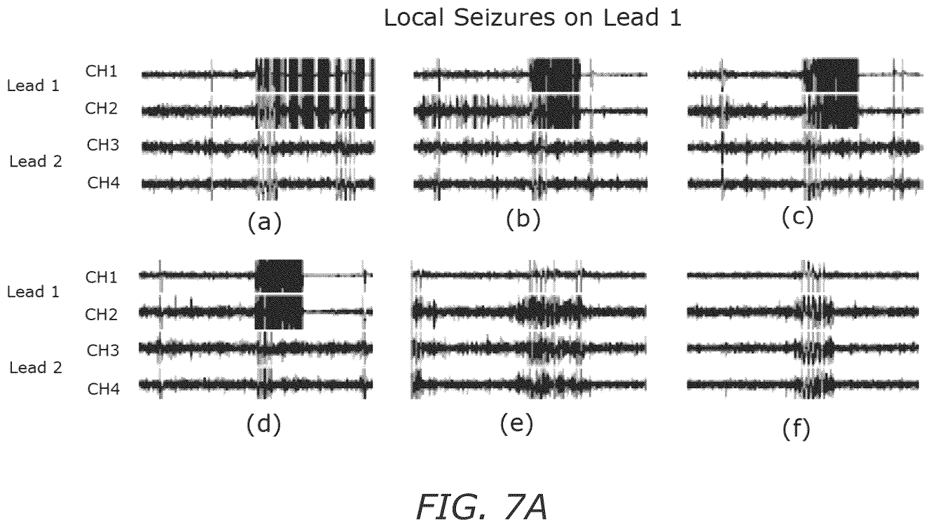

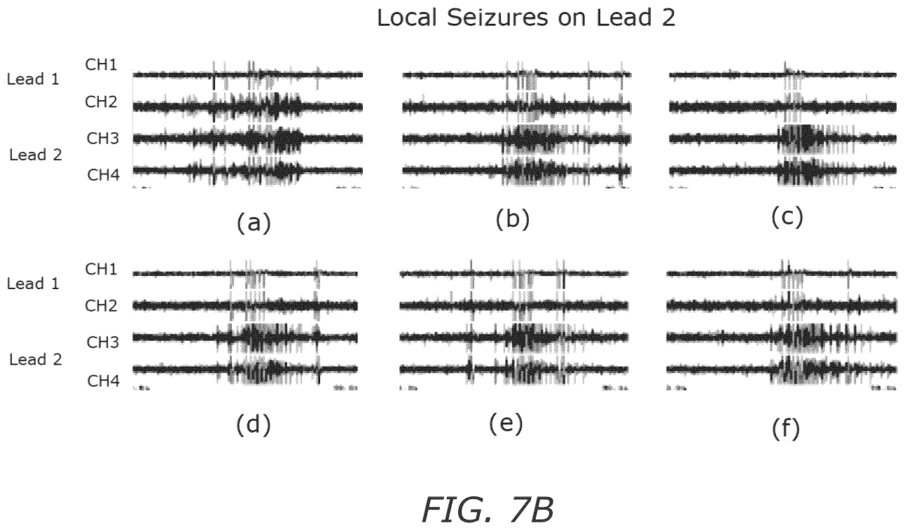

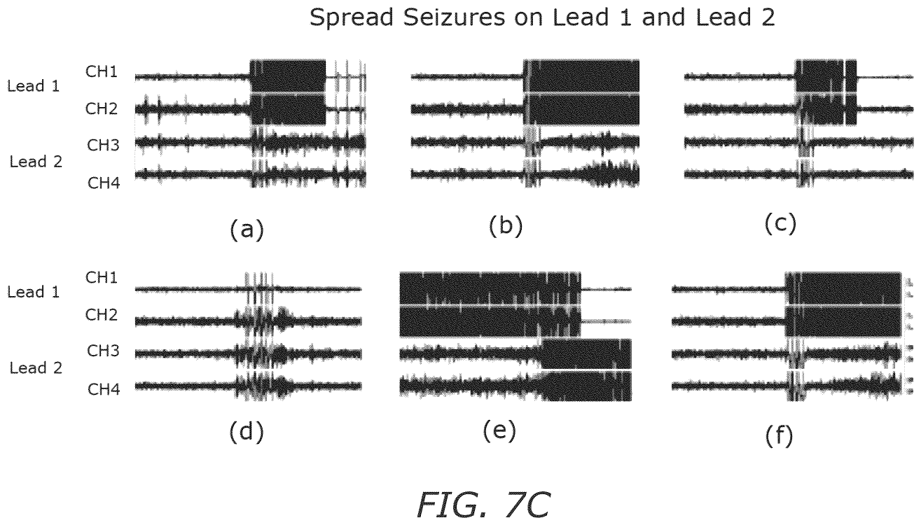

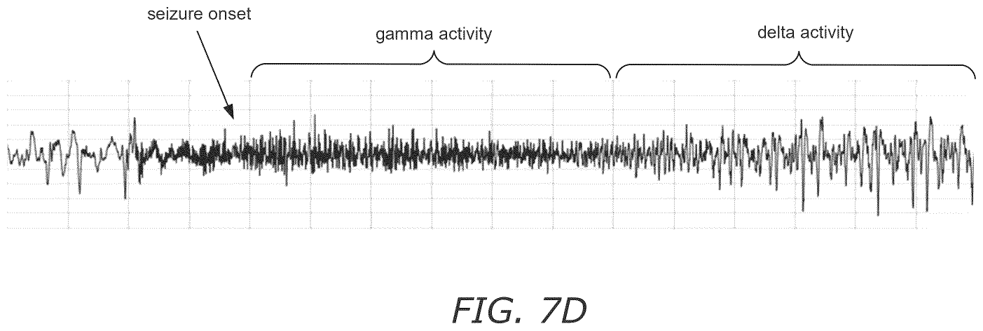

In some embodiments, the ATC module 603 is further configured to classify an EEG record as a particular activity type based on the spread time (i.e., the difference in seizure onset time on CH1/CH2 vs CH3/CH4) for that EEG record. For example, an EEG record with a spread time above a threshold value, e.g., 10 seconds, and brain activity frequency above a certain threshold, e.g., 50 Hz, is classified by the ATC module 603 as a low voltage fast activity pattern. As another example, if the spread time is below 5 seconds and the brain activity frequency is below 20 Hz, the ATC module 603 can classify it as theta onset pattern. With reference to FIG. 7A, the ATC module 603 can classify an EEG record of a seizure on lead 1 (CH1/CH2) as containing a first type of activity (type1-activity), such as semi-rhythmic beta type activity. An example of such an EEG record is shown in panel (e) of FIG. 7A. With reference to FIG. 7B, the ATC module 603 can classify an EEG record of a seizure on lead 2 (CH3/CH4) as containing a second type of activity (type2-activity), such as high amplitude beta activity. An example of such an EEG record is shown in panel (a) of FIG. 7B. With reference to FIG. 7C, the ATC module 603 can classify an EEG record of a seizure on lead 1 (CH1/CH2) and lead 2 (CH3/CH4) as containing an activity type 3, such as rhythmic theta. An example of such an EEG record is shown in panel (b) of FIG. 7C.

In some embodiments, the ATC module 603 is further configured to classify an EEG record as having a transition in activity type. For example, with reference to FIG. 7D, an electrographic seizure can start with primary gamma activity and transition to delta activity during the body of the seizure. In some embodiments, the ATC module 603 includes a submodule that determines an EEG record has a transition in activity type based on power spectral density (PSD) analysis. To this end, the ATC module 603 submodule computes PSDs for a moving window of the EEG record after the initial seizure onset to identify frequency peaks after seizure onset. If there are gamma peaks in a computed PSD, the portion of the EEG record within the window is still in the initial onset phase. An absence of gamma peaks along with a presence of only low frequency (e.g., delta) peaks in a computed PSD indicates the portion of the EEG record within the window is after the onset phase and includes the body of the seizure. An absence of peaks in the computed PSD indicates the portion of the EEG record within the window includes the end of the seizure (not shown in FIG. 7D).

Continuing with reference to FIGS. 5A and 6B, a set 610 of earliest onset EEG records 638 is applied to a grouping module 605 of the EO/AT module 505. The grouping module 605 is configured to group the earliest onset EEG records 638 in the set 610 by seizure onset type. For example, the earliest onset EEG records 638 in the set 610 is grouped into “n” different subgroups based on seizure onset types. The subgroups can include a set 612 of type1-activity earliest onset (EO) EEGs, a set 614 of type2-activity EO EEGs through to a set 616 of typen-activity EO EEGs. In an example, the type1-activity onset is a low voltage fast (LVF) seizure onset, while the type2-activity onset is a hypersynchronous seizure onset.

With continued reference to FIG. 6B, the set 612 of type1-activity EO EEGs, the set 614 of type2-activity EO EEGs through to the set 616 of typen-activity EO EEGs are applied to a selection module 607 of the EO/AT module 505. The selection module 607 is configured to separately process each set 612, 614, 616 of earliest onset EEG records to identify, for each set, the EEG record 613, 615, 617 with the earliest seizure onset time. Because the sets 612, 614, 616 of earliest onset EEGs records are grouped by seizure onset type, the selection module 607 in effect, identifies for each seizure onset type, the EEG record 613, 615, 617 with the earliest seizure onset time. In alternative embodiments, the selection module 607 is configured to separately process each set 612, 614, 616 of earliest onset EEG records to identify, for each set, a record 613, 615, 617 that is characteristic of that set in place of or in addition to having the earliest seizure onset time. For example, this may be a record whose location in a parameter space used for clustering by the ATC module 603 is nearest to the centroid of the cluster to which that record belongs, or a record whose likelihood of containing a certain activity type as determined by the ATC module 603 is higher than that of the other records in the set, or the first record available in the set, or a record having less artifact than the other records in the set.

With reference to FIGS. 5A and 6C, the detection window module 504 receives and separately processes the earliest onset EEG records 613, 615. For clarity of illustration, the earliest onset record 617 is not included in FIG. 6C. The detection window module 504 is configured to identify, for the type1-activity earliest onset EEG record 613, a first set 620 of regions of interest (ROIs) 630₁, 630₂, 630₃, each having a different length within a range of lengths, and to identify, for the type2-activity earliest onset EEG record 615, a second set 622 of ROIs 632₁, 632₂, 632₃, each having a different length within a range of lengths. These ROIs may also be referred to as detection windows. In some embodiments, the detection window module 504 is alternatively or additionally configured to identify sets of ROIs taken from multiple records. For example, the detection window module 504 may identify a first set 620 of ROIs where a single ROI, or a subset of ROIs having at least a first length and a second length, is drawn from each record in set 612 of type1-activity earliest onset (EO) EEGs. In this example, the detection window module 504 may also identify a second set 622 of ROIs where a single ROI, or a subset of ROIs having at least a first length and a second length, is drawn from each record in set 614 of type2-activity earliest onset (EO) EEGs.

In some embodiments, each ROI in a set of ROIs begins at the time of the seizure onset 636 within the earliest onset EEG record 613, 615. In some embodiments, the respective durations of ROI in a set of ROIs are based on programmed settings. For example, the duration or period of time of the first ROI 630₁, is d, the duration of the second ROI 630₂, is a×d, and the duration of the third ROI 630₃, is b×d, where variables a, b and d are predetermined. In some embodiments, the range of lengths is based on the seizure onset type of the earliest onset EEG record. Example ranges for ROI lengths are provided in Table 4 below:

TABLE 4

| Seizure Onset Type | ROI length range (in seconds) |
|---|---|
| Low Voltage Fast (LVF) | 1-2 |
| Hypersynchronous (Hypersync) | 2-4 |
| Attenuation (Atten) | 1-2 |
| Multiple (M) | 2-3 |
| Rhythmic Delta (D) | 2-4 |
| Rhythmic Theta (T) | 2-3 |
| Rhythmic Alpha (A) | 1.5-2.5 |
| Semi-Rhythmic Beta (SRB) | 1.5-2.5 |

With reference to FIGS. 5B and 6D, the candidate detection tool (CDT) module 506 is configured to determine a corresponding type1 candidate detection tool 624₁, 624₂, 624₃ based on each ROI 630₁, 630₂, 630₃ and a corresponding type2 candidate detection tool 626₁, 626₂, 626₃. based on each ROI 632₁, 632₂, 632₃. In some embodiments, the CDT module 506 is alternatively or additionally configured to determine a set of type1 candidate detection tools 624₁, 624₂, 624₃ based on ROIs taken from two or more records from set 612 of type1-activity earliest onset (EO) EEGs, and a corresponding set of type2 candidate detection tools 626₁, 626₂, 626₃ based on ROIs from two or more records from set 614 of type2-activity earliest onset (EO) EEGs. In some embodiments, the CDT module 506 is configured to determine one set of candidate detection tools 624₁, 624₂, 624₃ based on each ROI 630₁, 630₂, 630₃ for only one activity type; in some embodiments, the CDT module 506 is configured to determine more than two sets of candidate detection tools based on more than two activity types.

As an initial step in determining candidate detection tools, the CDT module 506 preprocesses each ROI to remove artifacts. The ROIs may have flat artifacts due to the brain implanted electrodes having dual tasks of sensing the EEG signal and delivering stimulation. Therefore, during those brief periods (hundreds of milliseconds) when the electrodes are not sensing the EEG signal, the neurostimulator records a flat artifact. In some embodiments, the CDT module 506 processes the ROI to remove these artifacts. In some embodiments, stimulation artifacts are removed by calculating when stimulation was delivered using EEG metadata, then removing the artifacts. In further embodiments, the artifact portions of the EEG signals that are removed are replaced with reconstructed brain activity. For example, machine learning models can be used to model and reconstruct brain activity data obscured by stimulation artifacts. See Menon K, Tcheng T, Seale C, Greene D, Morrell M and Desai SA (2025) Reconstructing signal during brain stimulation with Stim-BERT: a self-supervised learning model trained on millions of iEEG files. Front. Artif. Intell. 8:1502504. doi: 10.3389/frai.2025.1502504. This method enables the estimation and replacement of missing or corrupted time series segments with learned representations of brain activity, thereby preserving the integrity of the underlying brain signals. In some embodiments, the artifact removal or reconstruction methods described here may be applied prior to use of the CDT module 506, for example before records are presented to the electrographic seizure classification module 501, before records are presented to the EO/AT model 505, or before records are presented to the detection window module 504.

After preprocessing of the ROIs, each type1 ROI 630₁, 630₂, 630₃ is individually processed by the candidate detection tool (CDT) module 506 to determine a corresponding activity-type 1 candidate detection tool 624₁, 624₂, 624₃. Likewise, each type2-activity ROI 632₁, 632₂, 632₃ is individually processed by the CDT module 506 to determine a corresponding type2 candidate detection tool 626₁, 626₂, 626₃. The respective detection tools are specified by configuration information that includes detection-tool type, e.g., halfwave, line length, etc., and a detection parameter set.

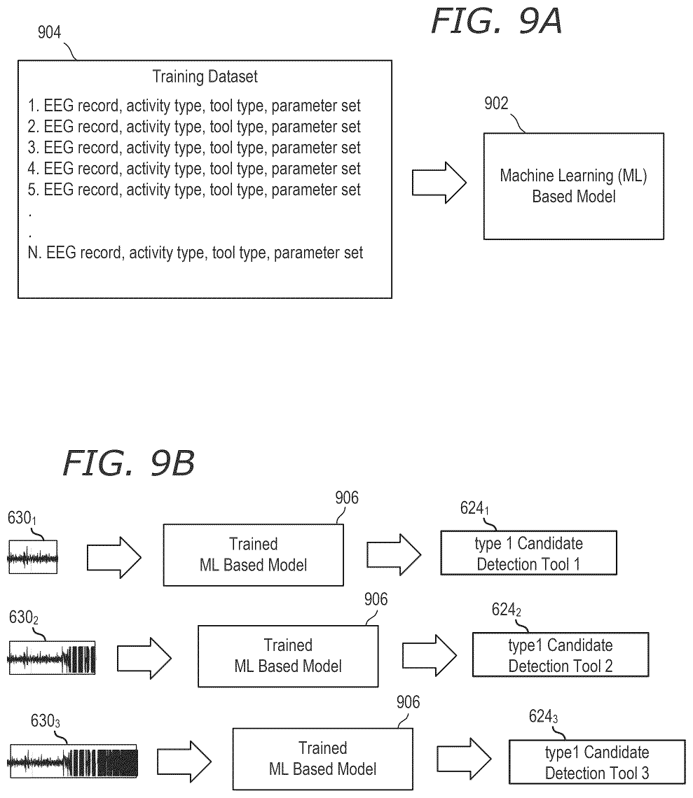

With reference to FIGS. 9A and 9B, in one embodiment, the CDT module 506 includes a machine learning based model 902 configured to determine candidate detection tools based on ROIs. The machine learning based model 902 is trained on a dataset 904 that includes EEG records with electrographic patterns or events that correspond to specific activity types, such as those listed in Table 4. The dataset also includes, for each EEG record, the activity type of the EEG record and information on the configuration of the detection tool that detected the pattern or event. The configuration information includes the type of detection tool and the detection parameter set of the detection tool. The configuration information can also include the size of the detection window. From this dataset 904, the machine learning based model 902 learns associations between electrographic patterns or events and activity types and detection tool configurations. In some embodiments, the machine learning based model 902 is of an architecture similar to the above disclosed ESC model.

With reference to FIG. 9B, the trained machine learning based model 906 is applied to each ROI 630₁, 630₂, 630₃, to provide a candidate detection tool 624₁, 624₂, 624₃ for that ROI, where each candidate detection tool is specified by configuration information, e.g., detection-tool type, detection parameter set, etc. Similarly, although not shown in FIG. 9B, the trained machine learning based model 906 is applied to each ROI 632₁, 632₂, 632₃, to provide a candidate detection tool 626₁, 626₂, 626₃ for that ROI.

In other embodiments, each type1 ROI 630₁, 630₂, 630₃ and each type2 ROI 632₁, 632₂, 632₃ is individually processed to determine a corresponding candidate parameter set, as disclosed in the following documents, each of which is hereby incorporated by reference: 1) U.S. Pat. No. 9,392,972, titled “Methods and Systems for Automatically Identifying Detection Parameters for an Implantable Medical Device,” 2) U.S. Pat. No. 10,722,176, titled “Methods and Systems for Automatically Identifying Detection Parameters for an Implantable Medical Device,” and 3) U.S. Pat. No. 10,729,352, titled “Neurological Event Detection Tools for Implantable Medical Devices.”

With reference to FIGS. 5B and 6E, the detection tool selection (DTS) module 508 receives and processes each type1-activity candidate detection tool 624₁, 624₂, 624₃ to obtain, for each candidate detection tool, metrics that represent the performance of the candidate detection tool for detecting electrographic activity corresponding to the type1-activity seizure onset. Similarly, the detection tool selection module 508 receives and processes each type2-activity candidate detection tool 626₁, 626₂, 626₃ to obtain, for each candidate detection tool, metrics that represent the performance of the candidate detection tool for detecting electrographic activity corresponding to the type2-activity seizure onset.

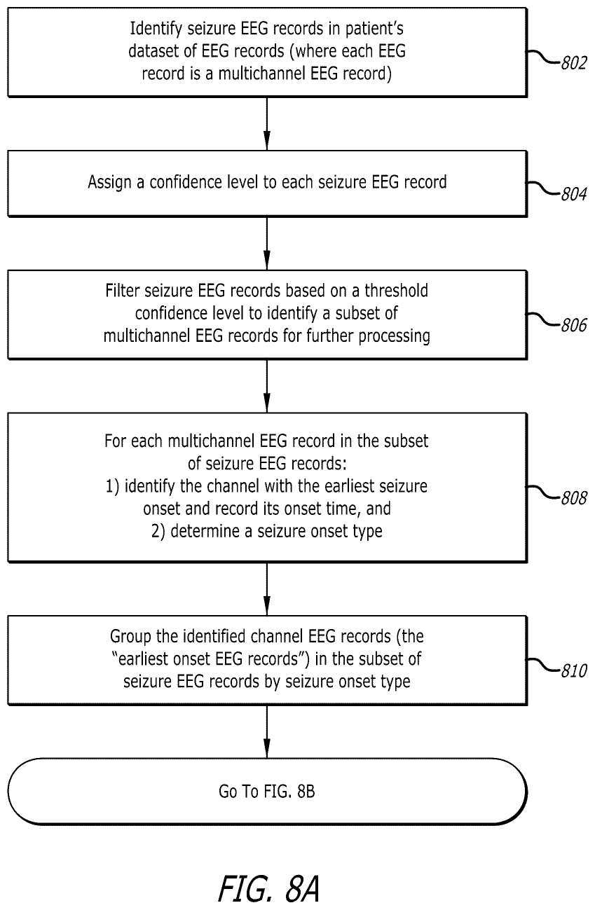

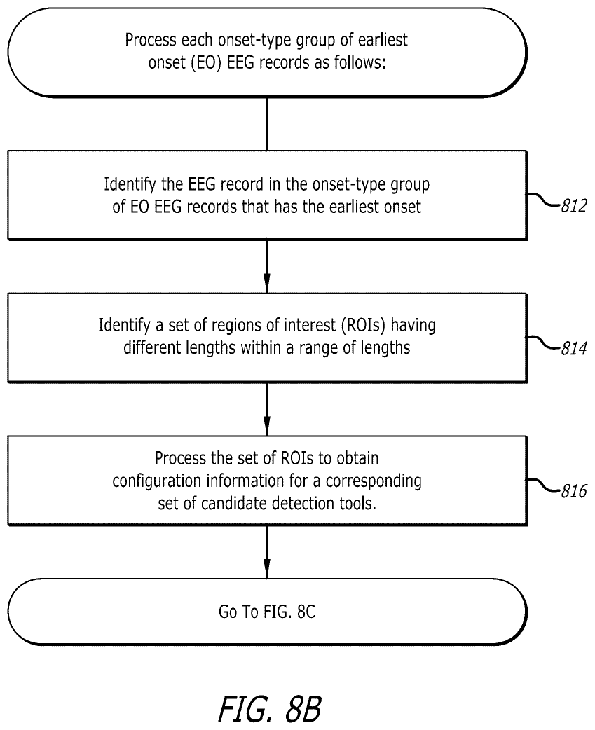

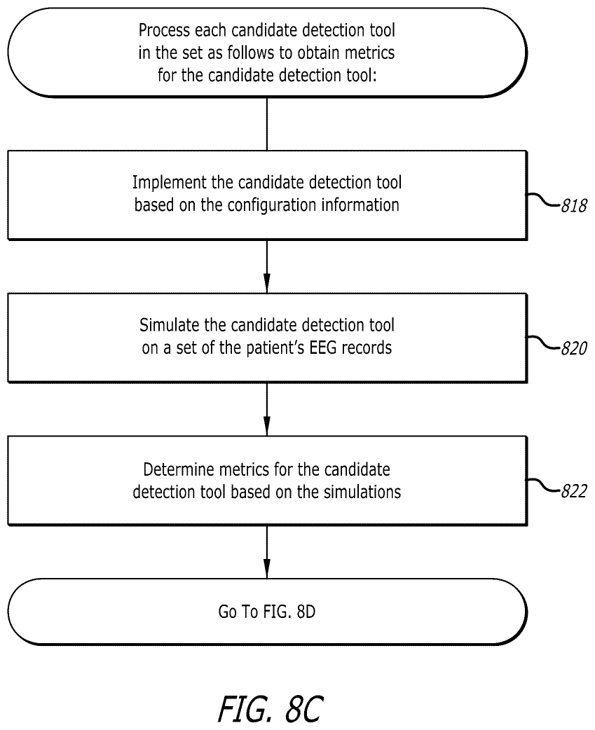

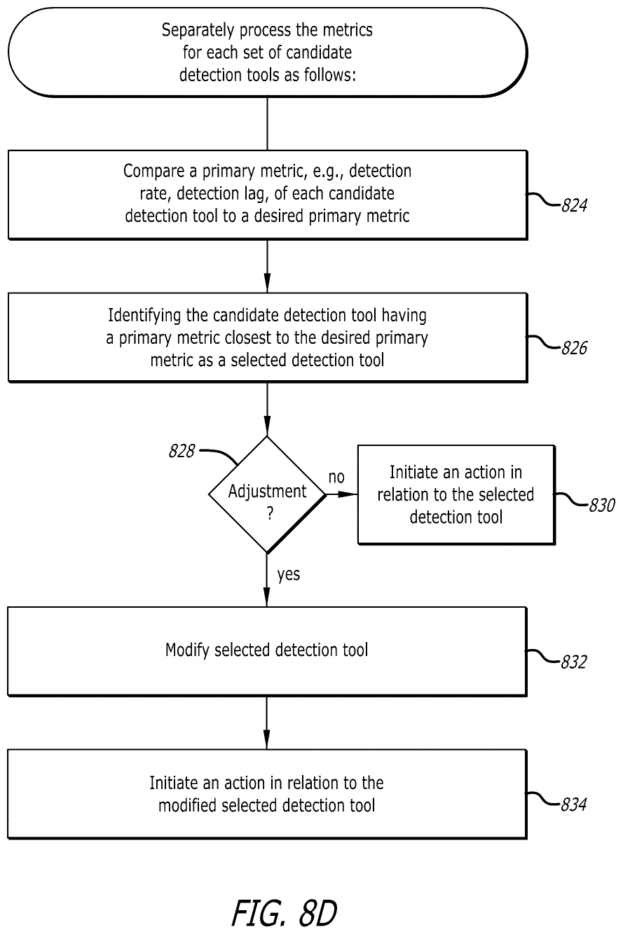

To these ends, the detection tool selection module 508 is configured to: 1) implement each candidate detection tool 624₁, 624₂, 624₃, 626₁, 626₂, 626₃ determined by the candidate detection tool module 506, 2) simulate each of the candidate detection tools by applying the tools to the same set of EEG records to obtain for each EEG record a simulation outcome, e.g., detections of abnormal activity, 3) determine a set of metrics for each candidate detection tool based on the simulation outcomes, and 4) process the respective metrics to identify a selected type1-activity detection tool 624 and a selected type2-activity detection tool 626. These functions of the detection tool selection module 508 are disclosed below with reference to FIG. 8C, blocks 818 through 822 and FIG. 8D, blocks 824 through 830.

Regarding simulations of the candidate detection tools, the detection tool selection module 508 applies each candidate detection tool 624₁, 624₂, 624₃, 626₁, 626₂, 626₃ to the same set of EEG records selected for simulation. In some embodiments, the set of EEG records selected for simulation is obtained from the EEG records dataset 510 and includes all of the EEG records recorded by the IMD of the patient, or a subset thereof. In some embodiments, the set of EEG records selected for simulation includes a specific number of the most recently recorded records, or the records recorded within a specified time period. In some embodiments, the set of EEG records selected for simulation includes EEG records across a patient population having characteristics, e.g., demographics, age, lead location, lead type, seizure morphology, onset zone, type of brain abnormality, etc., in common with the patient.

With continued reference to FIGS. 5B and 6E, after the detection tool selection module 508 identifies the selected detection tools 624, 626, it initiates an action in relation to the selected detection tool. In some embodiments, the action is implementing the selected detection tool 624, 626 in the patient's IMD. For example, as shown in FIG. 5B, an type1-activity detection tool 514 of the detection subsystem 326 of the IMD is implemented by programming a detection tool of the detection-tool type, e.g., halfwave detector, line length detector, specified by the configuration information 512 of the selected type1-activity detection tool 624 with the detection parameter set specified by the configuration information for the purposes of detecting type1-activity electrographic activity.

Similarly, a type2-activity detection tool 518 of the detection subsystem 326 of the IMD is implemented by programming a detection tool of the detection-tool type, e.g., halfwave detector, line length detector, specified by the configuration information 516 of the selected type2-activity detection tool 626 with the detection parameter set specified by the configuration information for the purposes of detecting type2-activity electrographic activity.

The type1-activity detection tool 514 can be coupled to a sensing channel that is known to sense type1 activity. The type2-activity detection tool 518 can be coupled to a sensing channel that is known to sense type2 activity. The type1-activity detection tool 514 and the type2-activity detection tool 518 can be coupled in parallel to a sensing channel that is known to sense electrical activity that includes a transition between type1 activity and type2 activity, such as shown in FIG. 7D.

With reference to FIG. 2, an alternative action of the detection proposer 202 is transmitting the configuration information 512 for type1 selected detection tool 624 and the configuration information 516 for the type2 selected detection tool 626 to a programmer 216 for storage and subsequent programming into the neurostimulator 302.

FIGS. 8A-8D are a flowchart of a method of processing electrical activity of a brain to propose one or more detection tools for an implantable medical device, where each detection tool is characterized by a detection-tool type and a set of detection parameters. In some embodiments, the method is performed by the detection proposer 202.

At block 802, and with reference to FIGS. 5A and 6A, an ESC model 501 is applied to each of a plurality of EEG records 602, 604 of a brain included in a set 600 of records. In some embodiments, each EEG record 602, 604 is a multichannel EEG record that includes a first channel record that corresponds to electrical activity sensed by a first sensing channel (CH1) of an implanted medical device, a second channel record that corresponds to electrical activity sensed by a second sensing channel (CH2) of the implanted medical device, a third channel record that corresponds to electrical activity sensed by a third sensing channel (CH3) of the implanted medical device, and a fourth channel record that corresponds to electrical activity sensed by a fourth sensing channel (CH4) of the implanted medical device. In some embodiments, the multichannel EEG record includes only two channels.

The ESC model 501 is configured to classify each of the plurality of multichannel EEG records 602, 604 as either a seizure record or a non-seizure record. As previously mentioned, in some embodiments the ESC model 501 is a machine learning based model trained in accordance with one of the models or a combination of models described in Barry et al. or Arcot Desai et al.

At block 804, a confidence level is assigned to each multichannel EEG record 602 that is classified as a seizure record. To this end, the ESC model 501 is further configured to assign a confidence level that represents the likelihood that a multichannel EEG record 602 classified as a seizure record is indeed a seizure record. For example, as mentioned previously, in some embodiments the ESC model 501 assigns a confidence level in the range of 0.1 to 0.9, where 0.1 indicates a low confidence level and 0.9 indicates a high confidence level. In other embodiments, the ESC model 501 assigns a confidence level in the range of 0.0 to 1.0, where 0.0 indicates the lowest confidence level and 1.0 indicates the highest confidence level.

At block 806, and with reference to FIGS. 5A and 6A, a set 606 of multichannel EEG records 602 classified as seizure records by the ESC model 501 is applied to a classification confidence filter 503. The classification confidence filter 503 is configured to filter the set 606 of multichannel seizure records 602 based on a threshold confidence level to provide a subset 608 of multichannel seizure records 602 for further processing. In some embodiments, the classification confidence filter 503 is programmed to include multichannel seizure records 602 with a confidence level at or above the threshold confidence level in the subset 608 of seizure records. The threshold confidence level is, for example, 0.8.

At block 808, and with reference to FIGS. 5A and 6B, models of an EO/AT module 505 are applied to each multichannel seizure record 602 in the subset 608 of seizure records to determine a seizure onset time 636a-d for each channel record 638a-d and to determine an activity type for the multichannel seizure record. Regarding FIG. 6B, for ease of illustration and description a single multichannel seizure record 602 is shown in detail. It is understood, however, that each multichannel seizure record 602 in the subset 608 of seizure records is processed by the EO/AT module 505.

Continuing with FIG. 6B, the SOD module 601 of the EO/AT module 505 is configured to be applied to each multichannel seizure record 602 in the subset 608 of seizure records, to determine a seizure onset time 636a-d for each channel record 638a-d and to identify the channel record with the earliest seizure onset, and to record the time of that earliest seizure onset. As previously mentioned, in some embodiments, the SOD module 601 comprises a convolutional neural network (CNN) based architecture. In other embodiments, the SOD module 601 is a deep learning model created using different methods, such as attention models (or other sequence based models) or signal processing algorithms. See, for example, “Recurrent Neural Networks for Forecasting Epileptiform Electrographic Activity 24 Hours in Advance,” AES abstract no. 2.050, published Nov. 6, 2018.

The ATC module 603 of the EO/AT module 505 is configured to classify, for each multichannel seizure record 602 in the subset 608 of seizure records, a type or pattern of seizure onset. Example of seizure onset types are provided above in Table 3. As previously mentioned, in some embodiments the ATC module 603 invokes a clustering method in which features are extracted from the EEG records and passed through a clustering algorithm such as Bayesian Gaussian Mixture Models as disclosed in Barry et al. to determine which type/pattern (or cluster) of seizure onset the EEG records falls within. The ATC module 603 is trained to recognize various type/patterns of electrical activity in EEG records corresponding to seizure onsets. Some examples are shown in the table below along with a description. EEG examples with these activity patterns are further described in Nune et al., “Treatment of drug-resistant epilepsy in patients with periventricular nodular heterotopia using RNS System: Efficacy and description of chronic electrophysiological recordings,” Clinical Neurophysiology 2019, which is incorporated by reference.

Thus, the SOD module 601 and the ATC module 603 function together to identify, for each multichannel seizure record 602 in the subset 608 of seizure records, the channel EEG record 638 with the earliest seizure onset 636 (referred to as earliest onset EEG record) and for each earliest onset EEG record, an activity type or pattern of seizure onset. The collection of earliest onset EEG records 638 identified by the SOD module 601 and the ATC module 603 form a set 610 of earliest onset EEG records.

At block 810, and with reference to FIG. 6B, the set 610 of earliest onset EEG records 638 is applied to a grouping module 605 of the EO/AT module 505. The grouping module 605 is configured to group the earliest onset EEG records 638 in the set 610 by seizure onset type. For example, the earliest onset EEG records 638 in the set 610 is grouped into “n” different subgroups based on seizure onset types. The subgroups includes a set 612 of type1-activity earliest onset (EO) EEGs, a set 614 of type2-activity EO EEGs through to a set 616 of type n EO EEGs. In one example, the type1-activity onset is a low voltage fast (LVF) seizure onset, while the type2-activity onset is a hypersynchronous seizure onset.

At block 812, and with continued reference to FIG. 6B, the set 612 of type1-activity EO EEGs, the set 614 of type2-activity EO EEGs through to the set 616 of type n EO EEGs are applied to a selection module 607 of the EO/AT module 505. The selection module 607 is configured to separately process each set 612, 614, 616 of earliest onset EEG records to identify, for each set, the EEG record 613, 615, 617 with the earliest seizure onset time. Because the sets 612, 614, 616 of earliest onset EEGs records are grouped by seizure onset type, the selection module 607 in effect, identifies for each seizure onset type, the EEG record 613, 615, 617 with the earliest seizure onset time. In other embodiments, in block 812, the selection module 607 is configured to separately process each set 612, 614, 616 of earliest onset EEG records to identify, for each set, an EEG record 613, 615, 617 which is characteristic of the set to which it belongs. In such embodiments, the selection module 607, in effect, identifies for each seizure onset type an EEG record 613, 615, 617 which is characteristic of that seizure onset type.

At block 814, and with reference to FIGS. 5B and 6C, the detection window module 504 receives and separately processes the earliest onset EEG records 613, 615. To this end, the detection window module 504 is configured to identify, for the type1-activity earliest onset EEG record 613, a first set 620 of regions of interest (ROIs) 630₁, 630₂, 630₃, each having a different length within a range of lengths, and to identify, for a type2-activity earliest onset EEG record 615, a second set 622 of ROIs) 632₁, 632₂, 632₃, each having a different length within a range of lengths. As disclosed above, in some embodiments, each ROI in a set of ROIs begins at the time of the seizure onset 636 within the earliest onset EEG record 613, 615. In some embodiments, the respective durations of ROI in a set of ROIs is based on programmed settings. In some embodiments, the range of lengths is based on the seizure onset type of the earliest onset EEG record.

At block 816, and with reference to FIGS. 5B and 6D, the candidate detection tool module 506 receives and processes the type1 earliest onset EEG record 613 based on the first set 620 of ROIs 630₁, 630₂, 630₃, to determine a set of type 1 candidate detection tools 624₁, 624₂, 624₃ corresponding in number to the number of ROIs 630₁, 630₂, 630₃. The candidate detection tool module 506 also receives and processes the type2 earliest onset EEG record 615 based on the second set 622 of 632₁, 632₂, 632₃ to determine a set of type 2 candidate detection tools 626₁, 626₂, 626₃ corresponding in number to the number of ROIs 632₁, 632₂, 632₃. The sets of candidate detection tools is determined using one of the approaches described above.

Proceeding to FIG. 8C, at blocks 818 through 822, and with reference to FIGS. 5B and 6E, the detection tool selection module 508 receives and processes each type1 candidate detection tool 624₁, 624₂, 624₃ to obtain, for each candidate detection tool, metrics that represent the performance of the candidate detection tool for detecting electrographic activity corresponding to the type1-activity seizure onset. Similarly, the detection tool selection module 508 receives and processes each type2 candidate detection tool 626₁, 626₂, 626₃ to obtain, for each candidate detection tool, metrics that represent the performance of the candidate detection tool for detecting electrographic activity corresponding to the type2-activity seizure onset.

More specifically, at block 818, the detection tool selection module 508 implements a corresponding candidate detection tool for each candidate detection tool based on the configuration information provided by the candidate detection tool module 506. To this end, the detection tool selection module 508 programs a detection tool of the type, e.g., half wave detection tool, line length detection tool etc., specified in the configuration information with the detection parameter set specified in the configuration information.

At block 820, for each candidate detection tool 624₁, 624₂, 624₃, 626₁, 626₂, 626₃, the detection tool selection module 508 simulates the candidate detection tool on a simulation set of EEG records. In some embodiments, the EEG records in the simulation set include all or a subset of the patient's EEG records that are available in the EEG records dataset 510.

At block 822, for each candidate detection tool 624₁, 624₂, 624₃, 626₁, 626₂, 626₃, the detection tool selection module 508 determines a set of metrics for the candidate detection tool based on the simulations. The set of metrics can include one or more of the following:

Detection rate—the number of abnormal activity detections within a time period for a given patient. For example, for a patient having an implanted neurostimulation system that is known to deliver 2-3 stimulations per minute in response to a detection of a seizure onset, the target measure of detection rate is about 1,500-2,500 detections/day. A measure of detection rate, e.g., detections per hour, detections per day, etc., is obtained by simulating the candidate detection tool on a set of EEG records (including both seizure EEG records and non-seizure EEG records) of the patient that span a 24 hour period.

Detection lag—the average of time delays determined for a candidate detection tool for a set of seizure EEG records, where an individual time delay is determined for each seizure EEG record as the difference between the time of detection of an abnormal activity event as determined by the candidate detection tool and the time of the actual seizure onset as determined by a seizure onset detection model.

True positive rate—the percentage of true abnormal activity detections made by the candidate detection tool out of the total number of simulations on seizure EEG records (i.e., EEG records that are known to include a seizure onset). Sensitivity is measured by simulating the candidate detection tool on multiple seizure EEG records from the patient. It is expressed as a percentage and is also referred to as the true positive rate, reflecting the accuracy of the tool in detecting actual abnormal activity patterns

False positive rate—the percentage of abnormal activity detections made by the candidate detection tool on non-seizure EEG records (i.e., EEG records known to not include a seizure onset). This rate is calculated by simulating the candidate detection tool on multiple non-seizure EEG records from the patient. It represents the proportion of false detections and reflects how often the tool incorrectly identifies normal activity as abnormal.

At block 824, the detection tool selection module 508 compares a primary metric of each candidate detection tool 624₁, 624₂, 624₃ in a first set of candidate detection tools to a desired primary metric for those candidate detection tools. The detection tool selection module 508 also compares a primary metric of each candidate detection tool 626₁, 626₂, 626₃ in a second set of candidate detection tools to a desired primary metric for those candidate detection tools. In some embodiments, the primary metric e.g., detection rate, detection lag, etc., and tolerance or measure of closeness is predetermined by the detection proposer 202. In some embodiments, the primary metric is input to the detection proposer 202 by a user through a user interface.

In some embodiments, the primary metric is a detection rate corresponding to a number of detections within a certain time period. In some embodiments, the primary metric is a detection lag corresponding to the difference between a time of detection of an event by the candidate detection tool and a time of detection of the event by a seizure onset detection model. In some embodiments the primary metric for the first set of candidate detection tools is the same as the primary metric for the second set of candidate detection tools. In some embodiments the primary metric for the first set of candidate detection tools is different from the primary metric for the second set of candidate detection tools.

At block 826, the detection tool selection module 508 identifies the candidate detection tool 624₁, 624₂, 624₃ having a primary metric that is closest to the desired primary metric as the selected type1-activity detection tool 624 for the first set of candidate detection tools. The detection tool selection module 508 also identifies the candidate detection tool 626₁, 626₂, 626₃ having a primary metric that is closest to the desired primary metric as the selected type2-activity detection tool 626 for the second set of candidate detection tools. In some embodiments, identification of a selected type1-activity detection tool 624 by the detection tool selection module 508 comprises generating a new selected type1-activity detection tool 624 but is not constrained to be identical to one of 624₁, 624₂, 624₃, for example by using an optimization method such as simulated annealing, linear optimization, a genetic algorithm, or a machine learning algorithm seeded with one or more of the candidate detection tools 624₁, 624₂, 624₃ to generate a type1-activity detection tool 624 which yields a primary metric that is even closer to the desired primary metric than any of the candidate detection tools 624₁, 624₂, 624₃. Similarly, in some embodiments, identification of a selected detection tool 626 by the detection tool selection module 508 comprises generating a selected type2-activity detection tool 626 that is not constrained to be identical to one of 626₁, 626₂, 626₃.

At block 828, the detection tool selection module 508 determines if an adjustment should be made to one or more of the selected type1-activity detection tool 624 or the selected type2-activity detection tool 626.

In some embodiments, the detection tool selection module 508 automatically determines that an adjustment should be made when the primary metric of a selected detection tool 624, 626 is not within a tolerance of the desired primary metric or does not satisfy a measure of closeness with the desired primary metric. For example, the primary metric can be a detection rate, e.g., number of detections per day, and the tolerance or measure of closeness can be a specified percentage, e.g., within 10%. In this case, the detection tool selection module 508 determines that a selected detection tool 624, 626 having a detection rate that is not within the specified percentage of the desired detection rate should be adjusted. In another example, the primary metric can be the detection lag and the tolerance or measure of closeness can be a specified percentage, e.g., within 10%. In this case, the detection tool selection module 508 determines that a selected detection tool 624, 626 having a detection lag that is not within the specified percentage of the desired detection lag should be adjusted.

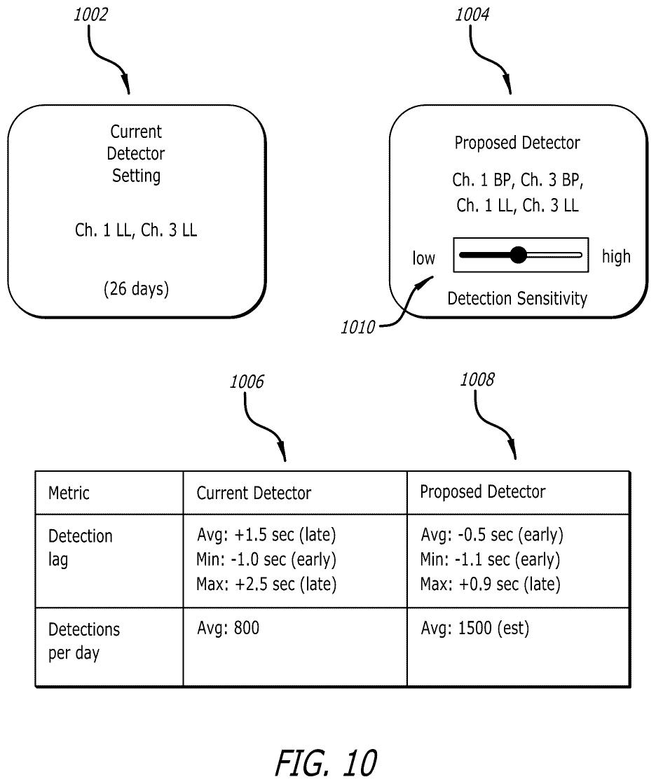

In some embodiments, the detection tool selection module 508 determines that an adjustment should be made based on receipt of a target primary metric through a user interface. In this case, the target primary metric replaces or superseded the desired primary metric and the detection tool selection module 508 performs an adjustment regardless of how close the primary metric of a selected detection tool 624, 626 is to the desired primary metric. With reference to FIG. 10, the detection proposer 202 is configured to present current detection tool configuration information 1002, selected detection tool configuration information 1004, and their respective performance metrics 1006, 1008 on a display, together with a user interface 1010 through which a user can input a target primary metric. In the example of FIG. 10, the user interface 1010 enables a user to input a target primary metric corresponding to a value for detection rate through a slide bar.

Continuing with block 828, if the detection tool selection module 508 determines no adjustment is needed for either of the selected type1-activity detection tool 624 or the selected type2-activity detection tool 626, the process proceeds to block 830, where the detection proposer 202 initiates an action in relation to each of the selected detection tools.

At block 830, in some embodiments, the detection proposer 202 directly programs the selected detection tools 624, 626 into an IMD. For example, with reference to FIG. 5B, the configuration information for the selected type1-activity detection tool 624 can be used to configure a type1-activity detection tool 514 in the detection subsystem 326 of the IMD for the purposes of detecting type1-activity electrographic activity. Similarly, the configuration information for the selected type2-activity detection tool 626 can be used to configure a type2-activity detection tool 518 in the detection subsystem 326 of the IMD for the purposes of detecting type2-activity electrographic activity.

At block 830, in some embodiments, the detection proposer 202 transmits the configuration information for the selected type1-activity detection tool 624 and the configuration information for the selected type2-activity detection tool 626 to a programmer 216 (shown in FIG. 2) for storage and subsequent approval by a doctor and programming into an IMD 206.

Returning to block 828, if the detection tool selection module 508 determines an adjustment should be made to one or more of the selected type1-activity detection tool 624 or the selected type2-activity detection tool 626, the process proceeds to block 832, where the detection tool selection module modifies one or more of the selected detection tools.

At block 832, in some embodiments the detection tool selection module 508 modifies a selected detection tool by adjusting one or more of the parameters in the detection parameter set for that detection tool, simulating the modified selected detection tool on the same simulation set of EEG records that the original selected detection tool was simulation on, and repeating the adjusting and simulating until the primary metric is satisfied.

For example, in the case of a selected detection tool corresponding to a half-wave (HW) detector having a detection parameter set comprising: a minimum frequency of 20.8 Hz (HW window size=220 ms, count criterion=10), a maximum frequency of 62.5 Hz (HW minimum width=4 ms), a HW minimum amplitude of 56 counts, a hysteresis of 32 counts, a minimum duration of 1.02 seconds (detect analysis window size=2048 msec, bandpass threshold=8), and a detection persistence of 1 second, the detection tool selection module 508 modifies the selected detection tool as follows:

In response to a need or user request to increase the detection rate, the detection tool selection module 508 reduces the HW minimum amplitude from 56 to 48 counts. This would lead to more qualified half-waves per unit time and both earlier detection (reduced detection lag) and more frequent detection (higher detection rate). Alternatively, the bandpass threshold could be lowered from 8 to 7, thus requiring the qualified half-waves to be present for a shorter period of time. This would also result in both earlier and more frequent detection. Neither of these strategies would affect the frequency sensitivity of the half-wave detector.

In response to a need or user request to decrease the detection rate, detection tool selection module 508 increases the HW minimum amplitude from 56 to 64 counts. This would lead to fewer qualified half-waves per unit time and both later detection (increased detection lag) and less frequent detection (lower detection rate). Alternatively, the bandpass threshold could be increased from 8 to 9, thus requiring the qualified half-waves to be present for a longer period of time. This would also result in both later and less frequent detection. Neither of these strategies would affect the frequency sensitivity of the half-wave detector.

In the case of a selected detection tool corresponding to a line length (LL) detector, having a detection parameter set comprising a detection threshold of 75%, a short-term trend of 2.05 seconds, a long-term trend of 2 minutes (window size=4096 msec, inter-sample interval=4096 msec, sample count=32), the detection tool selection module 508 modifies the selected detection tool as follows:

In response to a need or user request to increase the detection rate, the detection tool selection module 508 decreases the detection threshold from 75% to 62.5%. This would cause the LL detector to be triggered when the average LL during the short-term trend is greater than the long-term average LL by a smaller amount, resulting in both earlier detection and more frequent detection. Alternatively, the LL short-term trend could be reduced from 2.05 sec to 1.02 seconds. This would cause the LL detector to be triggered when the average short-term LL exceeds the detection threshold for a shorter period of time, resulting in both earlier and more frequent detection.

In response to a need or user request to decrease the detection rate, the detection tool selection module 508 increases the detection threshold from 75% to 87.5%. This would cause the LL detector to be triggered when the average LL during the short-term trend is greater than the long-term average LL by a greater amount, resulting in both later detection and less frequent detection. Alternatively, the LL short-term trend could be increased from 2.05 sec to 4.10 seconds. This would cause the LL detector to be triggered when the average short-term LL exceeds the detection threshold for a longer period of time, resulting in both later and less frequent detection.

In the case of a selected detection tool corresponding to an area-under-the-curve (Area) detector, having a detection parameter set that is the same as the LL detector example, i.e., a detection threshold of 75%, a short-term trend of 2.05 seconds, a long-term trend of 2 minutes (window size=4096 msec, inter-sample interval=4096 msec, sample count=32), the detection tool selection module 508 modifies the selected detection tool in the same manner as the LL detector.

In response to a need or user request to increase the detection rate, the detection tool selection module 508 decreases the detection threshold from 75% to 62.5%. This would cause the Area detector to be triggered when the average Area during the short-term trend is greater than the long-term average Area by a smaller amount, resulting in both earlier detection and more frequent detection. Alternatively, the Area short-term trend could be reduced from 2.05 sec to 1.02 seconds. This would cause the Area detector to be triggered when the average short-term Area exceeds the detection threshold for a shorter period of time, resulting in both earlier and more frequent detection.

In response to a need or user request to decrease the detection rate, the detection tool selection module 508 increases the detection threshold from 75% to 87.5%. This would cause the Area detector to be triggered when the average Area during the short-term trend is greater than the long-term average Area by a greater amount, resulting in both later detection and less frequent detection. Alternatively, the Area short-term trend could be increased from 2.05 sec to 4.10 seconds. This would cause the Area detector to be triggered when the average short-term Area exceeds the detection threshold for a longer period of time, resulting in both later and less frequent detection.

Additional information on adjusting detection parameter sets is provided in U.S. Pat. No. 8,131,352, titled “System and Method for Automatically Adjusting Detection Thresholds in a Feedback-controlled Neurological Event Detector,” which is incorporated by reference.

After modification of one or more of the selected detection tools 624, 626 is completed the process proceeds to block 834, where the detection proposer 202 initiates an action in relation to each of the modified selected detection tools.

At block 834, in some embodiments, the detection proposer 202 directly programs the modified selected detection tools into an IMD. In some embodiments, the detection proposer 202 transmits the configuration information for the modified selected detection tools to a programmer 216 (shown in FIG. 2) for storage and subsequent approval by a doctor and programming into an IMD 206.

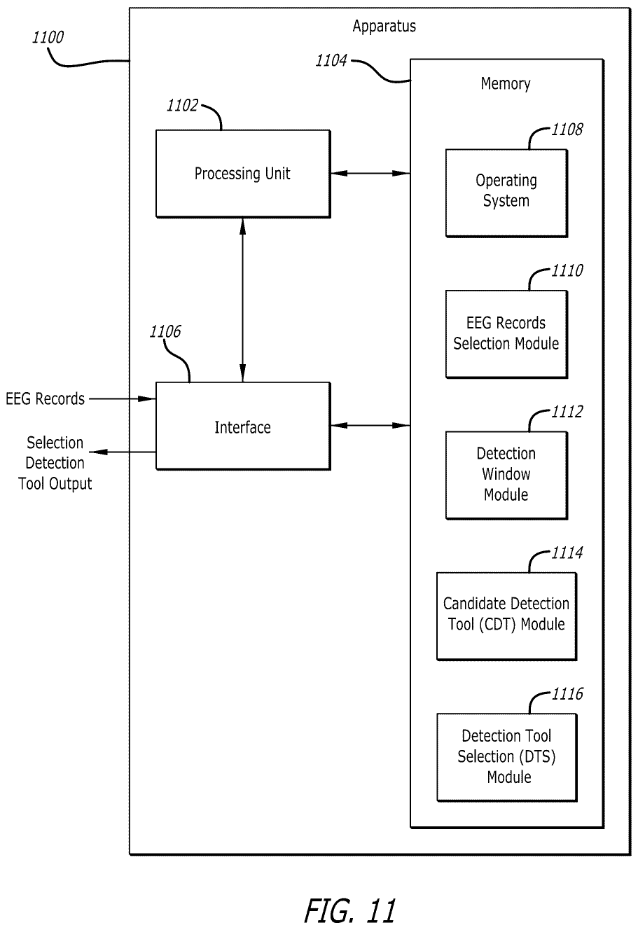

FIG. 11 is a schematic block diagram of an apparatus 1100 corresponding to the detection proposer 202 of FIG. 2. The apparatus 1100 is configured to execute instructions related to the detection set processes described above with reference to FIGS. 5A-8D. The apparatus 1100 is embodied in any number of processor-driven devices, including, but not limited to, a server computer, a personal computer, one or more networked computing devices, an application-specific circuit, a minicomputer, a microcontroller, and/or any other processor-based device and/or combination of devices.

The apparatus 1100 includes one or more processing units 1102 configured to access and execute computer-executable instructions stored in at least one memory 1104. The processing unit 1102 is implemented as appropriate in hardware, software, firmware, or combinations thereof. Software or firmware implementations of the processing unit 1102 can include computer-executable or machine-executable instructions written in any suitable programming language to perform the various functions described herein. The processing unit 1102 can include, without limitation, a central processing unit (CPU), a digital signal processor (DSP), a reduced instruction set computer (RISC) processor, a complex instruction set computer (CISC) processor, a microprocessor, a microcontroller, a field programmable gate array (FPGA), a System-on-a-Chip (SOC), or any combination thereof. The apparatus 1100 can also include a chipset (not shown) for controlling communications between the processing unit 1102 and one or more of the other components of the apparatus. The processing unit 1102 can also include one or more application-specific integrated circuits (ASICs) or application-specific standard products (ASSPs) for handling specific data processing functions or tasks.

The memory 1104 can include, but is not limited to, random access memory (RAM), flash RAM, magnetic media storage, optical media storage, and so forth. The memory 1104 can include volatile memory configured to store information when supplied with power and/or non-volatile memory configured to store information even when not supplied with power. The memory 1104 can store various program modules, application programs, and so forth that can include computer-executable instructions that upon execution by the processing unit 1102 can cause various operations to be performed. The memory 1104 can further store a variety of data manipulated and/or generated during execution of computer-executable instructions by the processing unit 1102.

The apparatus 1100 can further include one or more interfaces 1106 that can facilitate communication between the apparatus and one or more other apparatuses. For example, the interface 1106 is configured to receive EEG records from a database and to output information, e.g., therapy suggestions, to a display. Communication is implemented using any suitable structure or standard of communication. For example, communication with a database is through a LAN interface that implement protocols and/or algorithms that comply with various communication standards of the Institute of Electrical and Electronics Engineers (IEEE), such as IEEE 802.11. Communication with a user interface and display may be through wired or wireless means.

The memory 1104 can store various program modules, application programs, and so forth that can include computer-executable instructions that upon execution by the processing unit 1102 can cause various operations to be performed. For example, the memory 1104 can include an operating system module (O/S) 1108 that is configured to manage hardware resources such as the interface 1106 and provide various services to applications executing on the apparatus 1100.

The memory 1104 stores additional program modules such as: (1) an EEG records selection module 1110; (2) a detection window module 1112; (3) candidate detection tool (CDT) module 1114; and (4) a detection tool selection module 1116. Each of these modules includes computer-executable instructions that when executed by the processing unit 1102 cause various operations to be performed, including the operations described above with reference to FIGS. 5A-8D.

The apparatus 1100 and modules disclosed herein are implemented in hardware or software that is executed on a hardware platform. The hardware or hardware platform is a general purpose processor, a digital signal processor (DSP), an application specific integrated circuit (ASIC), a field programmable gate array (FPGA) or other programmable logic component, discrete gate or transistor logic, discrete hardware components, or any combination thereof, or any other suitable component designed to perform the functions described herein. A general-purpose processor is a microprocessor, but in the alternative, the processor is any conventional processor, controller, microcontroller, or state machine. A processor can also be implemented as a combination of computing components, e.g., a combination of a DSP and a microprocessor, a plurality of microprocessors, one or more microprocessors in conjunction with a DSP, or any other such configuration.

Systems and/or methods as described in this disclosure may use detection tools other than or in addition to half wave, line length, and area detection tools. Such detection tools may include, for example, detection tools based on the following parameters calculated from EEG data: root-mean-square (RMS) amplitude; amplifier saturation or other thresholding; aperiodic exponent; coherence; correlation; covariance; signal power calculated by fast Fourier transform, wavelet transform, or other means in a frequency band such as the delta, alpha, beta, or gamma band of EEG; or the ratio of signal power calculated in frequency bands such as power in the beta band of EEG divided by power in the gamma band of EEG. Such detection tools may identify a change in a calculated parameter, for instance by using the short-term and long-term trend mechanism described previously in this disclosure. Such detection tools may include recurrent neural network (RNN) models such as convolutional neural networks (CNNs), long short-term memory (LSTM) networks, vision transformers, or other models configured to detect abnormal electrographic activity while operating efficiently in a low-power, low-memory implantable embedded system.

In some embodiments, steps of the systems and methods described in this disclosure may be combined; for example, identification and filtering of seizure EEG records in blocks 802, 804, and 806 may be performed implicitly by a neural network which identifies seizure onset times and determines seizure onset type in block 808, for example by configuring the EO/AT model 505 used in block 808 to yield seizure onset channel, time, and activity type for records which have electrographic seizures, and a determination of no electrographic seizure for records which do not have electrographic seizures.

The various aspects of this disclosure are provided to enable one of ordinary skill in the art to practice the present invention. Various modifications to exemplary embodiments presented throughout this disclosure will be readily apparent to those skilled in the art. Thus, the claims are not intended to be limited to the various aspects of this disclosure, but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

What is claimed is:

1. A system for proposing a detection tool for an implanted medical device (IMD) of a patient, wherein the detection tool detects for an event in EEG signals sensed by the IMD, the system comprising:
a memory; and
a processor coupled to the memory and configured to:
apply a machine learning based model to a plurality of EEG records of the patient to identify a set of EEG records with electrographic activity indicative of an electrographic seizure, where the EEG records comprise a plurality of channel EEG signals sensed by a corresponding plurality of sensing channels of the IMD;
apply a machine learning based model to the identified set of EEG records to identify channel EEG signals having an earliest seizure onset; and
for each of the identified channel EEG signals:
process each of a plurality of regions of interest (ROI) of the identified channel EEG signal to implement a corresponding plurality of candidate detection tools to detect the event;
apply each candidate detection tool of the plurality of candidate detection tools to a simulation set of electrographic signals to determine a respective set of metrics; and
process the respective sets of metrics to identify a selected detection tool from among the candidate detection tools.

2. The system of claim 1, wherein the set of metrics comprises a primary metric and the processor processes the respective sets of metrics to identify a selected detection tool from among the candidate detection tools by being further configured to:
compare each primary metric to a desired primary metric; and
identify as the selected detection tool, the candidate detection tool having the primary metric closest to the desired primary metric.

3. The system of claim 2, wherein the primary metric is one of:
a detection rate corresponding to a number of event detections within a certain time period; and
a detection lag corresponding to a difference between a time of event detection by the candidate detection tool and a time of event detection by a seizure onset detection model.

4. The system of claim 2, further comprising:
determining to modify a detection parameter set of the selected detection tool such that the primary metric of the selected detection tool satisfies a criterion.

5. The system of claim 4, wherein the criterion comprises one of:
a measure of closeness between the desired primary metric and the primary metric of the selected detection tool, or
a target primary metric different from the desired primary metric.

6. The system of claim 5, further comprising a user interface, wherein the processor is further configured to:
receive, from the user interface, a user input corresponding to the target primary metric.

7. The system of claim 1, wherein the machine learning based model is trained to identify different EEG activity types and the identified channel EEG signals comprises at least two identified channel EEG signals, each corresponding to a different activity type.

8. The system of claim 1, wherein the machine learning based model is trained to identify different EEG activity types and the identified channel EEG signals comprises a channel EEG signal having a first portion corresponding to a first EEG activity type and a second portion corresponding to a second EEG activity type.

9. The system of claim 8, wherein the processor is configured to perform the processing, configuring, applying, and processing for each of the first portion and the second portion to identify a first selected detection tool for detecting the first EEG activity type and a second selected detection tool for detecting the second EEG activity type.

10. The system of claim 1, wherein the processor processes each of a plurality of ROIs of the identified channel EEG signal to implement a corresponding plurality of candidate detection tools by being further configured to apply a machine learning based model to each of the plurality of ROIs, wherein the machine learning based model is trained to provide configuration information for a candidate detection tool.

11. The system of claim 10, wherein the configuration information comprises a detection tool type and a detection parameter set for the detection tool type.

12. The system of claim 10, wherein the machine learning based model is trained on a dataset comprising a plurality of EEG recordings, and for each EEG recording, additional information comprising an EEG activity type of the EEG record, a type of the detection tool that detected a pattern in the EEG record indicative of the EEG activity type, and a detection parameter set for the detection tool.

13. The system of claim 1, wherein each ROI has a different duration from a time that is at or near a seizure onset.

14. The system of claim 13, wherein each different duration is in a range determined by an EEG activity type associated with the identified channel EEG signal.

15. The system of claim 1, wherein the simulation set of electrographic signals comprises one or more of:
seizure electrographic signals and non-seizure electrographic signals,
signals of the patient that were sensed and recorded by the IMD within a specified time range,
a specified number of signals that were most recently recorded by the IMD, and
signals of other patients with one or more of seizure morphology, lead location, and demographics in common with the patient.

16. A method of proposing a detection tool for an implanted medical device (IMD) of a patient, wherein the detection tool detects for an event in EEG signals sensed by the IMD, the method comprising:
applying a machine learning based model to a plurality of EEG records of the patient to identify a set of EEG records with electrographic activity indicative of an electrographic seizure, where the EEG records comprise a plurality of channel EEG signals sensed by a corresponding plurality of sensing channels of the IMD;
applying a machine learning based model to the identified set of EEG records to identify channel EEG signals having an earliest seizure onset; and
for each of the identified channel EEG signals:
processing each of a plurality of regions of interest (ROI) of the identified channel EEG signal to implement a corresponding plurality of candidate detection tools to detect the event;
applying each candidate detection tool of the plurality of candidate detection tools to a simulation set of electrographic signals to determine a respective set of metrics; and
processing the respective sets of metrics to identify a selected detection tool from among the candidate detection tools.

17. The method of claim 16, wherein the machine learning based model is trained to identify different EEG activity types and the identified channel EEG signals comprises at least two identified channel EEG signals, each corresponding to a different activity type.

18. The method of claim 16, wherein the machine learning based model is trained to identify different EEG activity types and the identified channel EEG signals comprises a channel EEG signal having a first portion corresponding to a first EEG activity type and a second portion corresponding to a second EEG activity type.

19. The method of claim 18, wherein the processing, configuring, applying, and processing are performed for each of the first portion and the second portion to identify a first selected detection tool for detecting the first EEG activity type and a second selected detection tool for detecting the second EEG activity type.

20. The method of claim 16, wherein processing each of a plurality of ROIs of the identified channel EEG signal to implement a corresponding plurality of candidate detection tools comprises applying a machine learning based model to each of the plurality of ROIs, wherein the machine learning based model is trained to provide configuration information for a candidate detection tool.

21. The method of claim 20, wherein the machine learning based model is trained on a dataset comprising a plurality of EEG recordings, and for each EEG recording, additional information comprising an EEG activity type of the EEG record, a type of the detection tool that detected a pattern in the EEG record indicative of the EEG activity type, and a detection parameter set for the detection tool.

22. The method of claim 16, wherein the set of metrics comprises a primary metric and processing the respective sets of metrics to identify a selected detection tool from among the candidate detection tools comprises:
comparing each primary metric to a desired primary metric; and
identifying as the selected detection tool, the candidate detection tool having the primary metric closest to the desired primary metric.

23. The method of claim 22, further comprising:
determining to modify a detection parameter set of the selected detection tool such that the primary metric of the selected detection tool satisfies a criterion.

24. The method of claim 16, further comprising:
initiating an action in relation to the selected detection tool, wherein the action comprises at least one of: implementing the selected detection tool in the IMD, and providing configuration information of the selected detection tool to a programmer.

25. An implantable medical device comprising:
a sensing channel configured to sense electrical activity;
a detection tool configured to detect an event in sensed electrical activity, wherein the detection tool is proposed by the system of claim 1; and
a therapy subsystem configured to output a stimulation therapy in response to a detection of the event by the detection tool.
